SEQUENCE LISTING (20 sequences)

SEQ ID NO: 1 - protein, 9 aa, Homo sapiens
EEQYNSTYR

SEQ ID NO: 2 - protein, 9 aa, Homo sapiens
EEQFNSTFR

SEQ ID NO: 3 - protein, 9 aa, Homo sapiens
EEQFNSTFR

SEQ ID NO: 4 - protein, 9 aa, Homo sapiens
EEQFNSTYR

SEQ ID NO: 5 - protein, 398 aa, Homo sapiens
MRLREPLLSGSAAMPGASLQRACRLLVAVCALHLGVTLVYYLAGRDLSRLPQLVGVSTPLQGGSNSAAAIGQSSGELRTGGARPPPPLGASSQPRPGGDSSPVVDSGPGPASNLTSVPVPHTTALSLPACPEESPLLVGPMLIEFNMPVDLELVAKQNPNVKMGGRYAPRDCVSPHKVAIIIPFRNRQEHLKYWLYYLHPVLQRQQLDYGIYVINQAGDTIFNRAKLLNVGFQEALKDYDYTCFVFSDVDLIPMNDHNAYRCFSQPRHISVAMDKFGFSLPYVQYFGGVSALSKQQFLTINGFPNNYWGWGGEDDDIFNRLVFRGMSISRPNAVVGRCRMIRHSRDKKNEPNPQRFDRIAHTKETMLSDGLNSLTYQVLDVQRYPLYTQITVDIGTPS

SEQ ID NO: 6 - protein, 385 aa, Homo sapiens
MPGASLQRACRLLVAVCALHLGVTLVYYLAGRDLSRLPQLVGVSTPLQGGSNSAAAIGQSSGELRTGGARPPPPLGASSQPRPGGDSSPVVDSGPGPASNLTSVPVPHTTALSLPACPEESPLLVGPMLIEFNMPVDLELVAKQNPNVKMGGRYAPRDCVSPHKVAIIIPFRNRQEHLKYWLYYLHPVLQRQQLDYGIYVINQAGDTIFNRAKLLNVGFQEALKDYDYTCFVFSDVDLIPMNDHNAYRCFSQPRHISVAMDKFGFSLPYVQYFGGVSALSKQQFLTINGFPNNYWGWGGEDDDIFNRLVFRGMSISRPNAVVGRCRMIRHSRDKKNEPNPQRFDRIAHTKETMLSDGLNSLTYQVLDVQRYPLYTQITVDIGTPS

SEQ ID NO: 7 - protein, 357 aa, Homo sapiens
MRLREPLLSGSAAMPGASLQRACRLLVAVCALHLGVTLVYYLAGRDLSRLPQLVGVSTPLQGGSNSAAAIGQSSGELRTGGARPPPPLGASSQPRPGGDSSPVVDSGPGPASNLTSVPVPHTTALSLPACPEESPLLVGPMLIEFNMPVDLELVAKQNPNVKMGGRYAPRDCVSPHKVAIIIPFRNRQEHLKYWLYYLHPVLQRQQLDYGIYVINQAGDTIFNRAKLLNVGFQEALKDYDYTCFVFSDVDLIPMNDHNAYRCFSQPRHISVAMDKFGFRLVFRGMSISRPNAVVGRCRMIRHSRDKKNEPNPQRFDRIAHTKETMLSDGLNSLTYQVLDVQRYPLYTQITVDIGTPS

SEQ ID NO: 8 - protein, 225 aa, Homo sapiens
MRLREPLLSGSAAMPGASLQRACRLLVAVCALHLGVTLVYYLAGRDLSRLPQLVGVSTPLQGGSNSAAAIGQSSGELRTGGARPPPPLGASSQPRPGGDSSPVVDSGPGPASNLTSVPVPHTTALSLPACPEESPLLVGPMLIEFNMPVDLELVAKQNPNVKMGGRYAPRDCVSPHKVAIIIPFRNRQEHLKYWLYYLHPVLQRQQLDYGIYVINQYEKIRRLLW

SEQ ID NO: 9 - protein, 24 aa, Homo sapiens
MRLREPLLSGSAAMPGASLQRACR

SEQ ID NO: 10 - protein, 20 aa, Homo sapiens
LLVAVCALHLGVTLVYYLAG

SEQ ID NO: 11 - protein, 354 aa, Homo sapiens
RDLSRLPQLVGVSTPLQGGSNSAAAIGQSSGELRTGGARPPPPLGASSQPRPGGDSSPVVDSGPGPASNLTSVPVPHTTALSLPACPEESPLLVGPMLIEFNMPVDLELVAKQNPNVKMGGRYAPRDCVSPHKVAIIIPFRNRQEHLKYWLYYLHPVLQRQQLDYGIYVINQAGDTIFNRAKLLNVGFQEALKDYDYTCFVFSDVDLIPMNDHNAYRCFSQPRHISVAMDKFGFSLPYVQYFGGVSALSKQQFLTINGFPNNYWGWGGEDDDIFNRLVFRGMSISRPNAVVGRCRMIRHSRDKKNEPNPQRFDRIAHTKETMLSDGLNSLTYQVLDVQRYPLYTQITVDIGTPS

SEQ ID NO: 12 - protein, 290 aa, Artificial Sequence
  B4GalT
GPASNLTSVPVPHTTALSLPACPEESPLLVGPMLIEFNMPVDLELVAKQNPNVKMGGRYAPRDCVSPHKVAIIIPFRNRQEHLKYWLYYLHPVLQRQQLDYGIYVINQAGDTIFNRAKLLNVGFQEALKDYDYTCFVFSDVDLIPMNDHNAYRCFSQPRHISVAMDKFGFSLPYVQYFGGVSALSKQQFLTINGFPNNYWGWGGEDDDIFNRLVFRGMSISRPNAVVGRCRMIRHSRDKKNEPNPQRFDRIAHTKETMLSDGLNSLTYQVLDVQRYPLYTQITVDIGTPS

SEQ ID NO: 13 - protein, 308 aa, Artificial Sequence
  B4GalT
GSSPLLDMGPASNLTSVPVPHTTALSLPACPEESPLLVGPMLIEFNMPVDLELVAKQNPNVKMGGRYAPRDCVSPHKVAIIIPFRNRQEHLKYWLYYLHPVLQRQQLDYGIYVINQAGDTIFNRAKLLNVGFQEALKDYDYTCFVFSDVDLIPMNDHNAYRCFSQPRHISVAMDKFGFSLPYVQYFGGVSALSKQQFLTINGFPNNYWGWGGEDDDIFNRLVFRGMSISRPNAVVGRCRMIRHSRDKKNEPNPQRFDRIAHTKETMLSDGLNSLTYQVLDVQRYPLYTQITVDIGTPSPRDHHHHHHH

SEQ ID NO: 14 - protein, 406 aa, Homo sapiens
MIHTNLKKKFSCCVLVFLLFAVICVWKEKKKGSYYDSFKLQTKEFQVLKSLGKLAMGSDSQSVSSSSTQDPHRGRQTLGSLRGLAKAKPEASFQVWNKDSSSKNLIPRLQKIWKNYLSMNKYKVSYKGPGPGIKFSAEALRCHLRDHVNVSMVEVTDFPFNTSEWEGYLPKESIRTKAGPWGRCAVVSSAGSLKSSQLGREIDDHDAVLRFNGAPTANFQQDVGTKTTIRLMNSQLVTTEKRFLKDSLYNEGILIVWDPSVYHSDIPKWYQNPDYNFFNNYKTYRKLHPNQPFYILKPQMPWELWDILQEISPEEIQPNPPSSGMLGIIIMMTLCDQVDIYEFLPSKRKTDVCYYYQKFFDSACTMGAYHPLLYEKNLVKHLNQGTDEDIYLLGKATLPGFRTIHC

SEQ ID NO: 15 - protein, 175 aa, Homo sapiens
MNSQLVTTEKRFLKDSLYNEGILIVWDPSVYHSDIPKWYQNPDYNFFNNYKTYRKLHPNQPFYILKPQMPWELWDILQEISPEEIQPNPPSSGMLGIIIMMTLCDQVDIYEFLPSKRKTDVCYYYQKFFDSACTMGAYHPLLYEKNLVKHLNQGTDEDIYLLGKATLPGFRTIHC

SEQ ID NO: 16 - protein, 9 aa, Homo sapiens
MIHTNLKKK

SEQ ID NO: 17 - protein, 17 aa, Homo sapiens
FSCCVLVFLLFAVICVW

SEQ ID NO: 18 - protein, 380 aa, Homo sapiens
KEKKKGSYYDSFKLQTKEFQVLKSLGKLAMGSDSQSVSSSSTQDPHRGRQTLGSLRGLAKAKPEASFQVWNKDSSSKNLIPRLQKIWKNYLSMNKYKVSYKGPGPGIKFSAEALRCHLRDHVNVSMVEVTDFPFNTSEWEGYLPKESIRTKAGPWGRCAVVSSAGSLKSSQLGREIDDHDAVLRFNGAPTANFQQDVGTKTTIRLMNSQLVTTEKRFLKDSLYNEGILIVWDPSVYHSDIPKWYQNPDYNFFNNYKTYRKLHPNQPFYILKPQMPWELWDILQEISPEEIQPNPPSSGMLGIIIMMTLCDQVDIYEFLPSKRKTDVCYYYQKFFDSACTMGAYHPLLYEKNLVKHLNQGTDEDIYLLGKATLPGFRTIHC

SEQ ID NO: 19 - protein, 320 aa, Artificial Sequence
  St6Gal
AKPEASFQVWNKDSSSKNLIPRLQKIWKNYLSMNKYKVSYKGPGPGIKFSAEALRCHLRDHVNVSMVEVTDFPFNTSEWEGYLPKESIRTKAGPWGRCAVVSSAGSLKSSQLGREIDDHDAVLRFNGAPTANFQQDVGTKTTIRLMNSQLVTTEKRFLKDSLYNEGILIVWDPSVYHSDIPKWYQNPDYNFFNNYKTYRKLHPNQPFYILKPQMPWELWDILQEISPEEIQPNPPSSGMLGIIIMMTLCDQVDIYEFLPSKRKTDVCYYYQKFFDSACTMGAYHPLLYEKNLVKHLNQGTDEDIYLLGKATLPGFRTIHC

SEQ ID NO: 20 - protein, 339 aa, Artificial Sequence
  ST6Gal
GSSPLLDMLEHHHHHHHHMAKPEASFQVWNKDSSSKNLIPRLQKIWKNYLSMNKYKVSYKGPGPGIKFSAEALRCHLRDHVNVSMVEVTDFPFNTSEWEGYLPKESIRTKAGPWGRCAVVSSAGSLKSSQLGREIDDHDAVLRFNGAPTANFQQDVGTKTTIRLMNSQLVTTEKRFLKDSLYNEGILIVWDPSVYHSDIPKWYQNPDYNFFNNYKTYRKLHPNQPFYILKPQMPWELWDILQEISPEEIQPNPPSSGMLGIIIMMTLCDQVDIYEFLPSKRKTDVCYYYQKFFDSACTMGAYHPLLYEKNLVKHLNQGTDEDIYLLGKATLPGFRTIHC

(19) United States
(12) Patent Application Publication
(10) Pub. No.: US 2023/0193339 A1
(43) Pub. Date: Jun. 22, 2023

(54) Preparation and Purification of Hypersialylated IGG

(71) Applicant: Momenta Pharmaceuticals, Inc., Titusville, NJ (US)

(72) Inventors: John Schaeck, Titusville, NJ (US); Steffen Graham, Titusville, NJ (US); Radouane Zouaoui, Titusville, NJ (US)

(21) Appl. No.: 17/926,102

(22) PCT Filed: May 19, 2021
(86) PCT No.: PCT/US2021/033156

§ 371 Date: Nov. 17, 2022

Related U.S. Application Data
(60) Provisional application No. 63/026,875, filed on May 19, 2020.

(51) Int. Cl. C12P 21/00; C07K 16/06; C07K 1/18; C07K 1/34; C07K 1/36

(52) U.S. Cl. CPC C12P 21/005; C07K 1/18; C07K 1/34; C07K 1/36; C07K 16/065; C07K 2317/41

(57) ABSTRACT

Methods for preparing and purifying hypersialylated IgG are described.

### CLAIM OF PRIORITY

This application claims the benefit of U.S. Provisional Application Serial No. 63/026,875, filed on May 19, 2020. The entire contents of the foregoing are incorporated herein by reference.

### TECHNICAL FIELD

The present disclosure relates to methods for preparing and purifying hypersialylated IgG.

### BACKGROUND

Intravenous immunoglobulin (IVIg), which is prepared from the pooled plasma of human donors (e.g., pooled plasma from at least 1,000 donors), is used to treat a variety of inflammatory disorders. However, IVIg preparations have distinct limitations, such as variable efficacy, clinical risks, high costs, and finite supply. Different IVIg preparations are frequently treated as interchangeable products clinically, but it is well-known that significant differences in product preparations exist that may impact tolerability and activity in selected clinical applications. At the current maximal dosing regimens, only partial and unsustained responses are obtained in many instances. In addition, the long infusion times (4-6 h) associated with the high volume of IVIg treatment consume significant resources at infusion centers and negatively affect patient-reported outcomes, such as convenience and quality of life.

The identification of the important anti-inflammatory role of Fc domain sialylation has presented an opportunity to develop more potent immunoglobulin therapies. Commercially available IVIg preparations generally exhibit low levels of sialylation on the Fc domain of the antibodies present. Specifically, they exhibit low levels of di-sialylation of the branched glycans on the Fc region.

Washburn et al. (Proceedings of the National Academy of Sciences, USA 112: E1297-E1306 (2015)) describes a controlled sialylation process to generate highly tetra-Fc-sialylated IVIg and showed that the process yields a product with consistent enhanced anti-inflammatory activity.

### SUMMARY

Thus, described herein are methods for preparing immunoglobulin G (IgG) having a very high level of Fc sialylation, particularly disialylation (sialylation on both the alpha 1,3 branch and the alpha 1,6 branch of the glycan at Asn297 (EU Numbering). The methods described herein can provide hypersialylated IgG (hsIgG) in which greater than 70% of the branched glycans on the Fc domain are sialylated on both branches (i.e., on the alpha 1,3 branch and the alpha 1,6 branch). HsIgG contains a diverse mixture of IgG antibodies, primarily IgG1 antibodies. The diversity of the antibodies is high. The immunoglobulins used to prepare hsIgG can be obtained, for example from pooled human plasma (e.g., pooled plasma from at least 1,000 – 30,000 donors). The immunoglobulins can be obtained from IVIg, including commercially available IVIg. HsIgG has far higher level of sialic acid on the branched glycans on the Fc region than does IVIg. This results in a composition that differs from IVIg in both structure and activity. HsIgG can be prepared as described in WO2014/179601 or Washbum et al. (Proceedings of the National Academy of Sciences, USA 112: E1297-E1306 (2015)), both of which are hereby incorporated by reference.

Described herein are improved methods for preparing and purifying hsIgG. The methods generate an unpurified hsIgG composition from which both purified hsIgG and, in some embodiments, the enzymes used to generate to prepare the hsIgG can be isolated. In some cases, the isolated enzymes can be reused.

Described herein are methods of producing a purified hypersialylated IgG (hsIgG) composition with greater than 75% of the branched Fc glycans on the hsIgG have a sialic acid on both the α1,3 branch and the α1,6 branch comprising: (a) providing a hsIgG composition comprising hsIgG and ST6Gal or an enzymatically active portion thereof; (b) diluting the composition in a citrate buffer at about 50 mM, about pH 4.5, thereby producing a buffered antibody composition; (c) applying the buffered antibody composition to a chromatography column comprising a resin with a sulfonic acid functional group under conditions that bind the hsIgG as well as the ST6Gal or enzymatically active portion thereof; and (d) selectively eluting the IgG from the column, thereby producing a purified hsIgG composition with greater than 75% of the branched Fc glycans on the hsIgG have a sialic acid on both the α1,3 branch and the α1,6 branch

In some embodiments, the step of providing a hsIgG composition comprises providing a composition comprising hsIgG and diluting the composition in 5X PBS at about a 1:1 dilution.

In some embodiments, the CEX column comprises a resin having a SO₃- functional group.

In some embodiments, selectively eluting the hsIgG comprises eluting in a buffer comprising about 400 mM or more NaCl.

In some embodiments, the buffer is citrate buffer at about 50 mM and about pH 4.5.

In some embodiments, the method does not contain any additional filtering, fractionation, or purification steps other than (a) and (b) before carrying out step (c).

In some embodiments, the method comprises a single depth filtering step, in addition to steps (a) and (b) before step (c).

In some embodiments, the additional depth filtering step is carried out between steps (a) and (c).

In some embodiments, the method does not comprise any additional filtering, fractionation, or purification steps beyond the single depth filtration step before carrying out step (c).

In some embodiments, the method comprises a viral inactivation step before step (c).

In some embodiments, the applying in step (c) is spaced out over 1, 2, or 3 applications.

In some embodiments, step (e) is repeated one or more times.

In some embodiments, step (c) is carried out at resident time(s) of from or from about 1, 2, 3, 4, 5, or 6 minutes.

In some embodiments the method further comprises, following step (e), one or more of: (f) blue dye chromatography (g) DE pad filtration step, (h) depth filtration step or (g) PS20 spiking & filtration step.

In some embodiments, the purified hsIgG composition comprises 80%, 85%, 90%, or 95% or more of the amount of unpurified hsIgG from the composition of step (a).

In some embodiments, the purified hsIgG composition comprises 100 ppm, 90 ppm, 80 ppm, 70 ppm, 60 ppm, 50 ppm, 40 ppm, or 30 ppm or less ST6Gal or enzymatically active portion thereof.

Also provided herein is a method of producing a purified hypersialylated IgG (hsIgG) composition comprising: (a) providing a hsIgG composition comprising hsIgG and ST6Gal or an enzymatically active portion thereof and optionally quenching the reaction; (b) diluting the composition in a buffer suitable for use with the column, thereby producing a buffered antibody composition; (c) applying the buffered antibody composition to a blue dye column, e.g., a blue column described herein, under conditions that allow 80% or more of the hsIgG to flow through, but allows only 100 ppm or less of the ST6 or enzymatically active portion thereof to flow through, thereby producing purified hsIgG.

In some embodiments, the blue dye chromatography column is a Trisacryl Blue (TABS) column and the buffer suitable for use with the column is citrate buffer, about 100 mM, about pH 4.5, with about 800 mM NaCl.

In some embodiments, the blue dye chromatography column is a Capto™ Blue or Capto™ Blue HS column and the buffer is about 250 mM glycine with about 800 mM NaCl at about pH 4.5.

In some embodiments, the step of providing a hsIgG composition comprises providing a composition comprising hsIgG and diluting the composition in 5X PBS at about a 1:1 dilution.

In some embodiments, the method does not contain any additional filtering, fractionation, or purification steps other than (a) and (b) before carrying out step (c).

In some embodiments, the method comprises a single depth filtering step, in addition to steps (a) and (b) before step (c).

In some embodiments, the additional filtering step is carried out between steps (a) and (c).

In some embodiments, the method does not comprise any additional filtering, fractionation, or purification steps beyond the single depth filtration step before carrying out step (c).

In some embodiments, the method comprises a viral inactivation step before step (c).

In some embodiments, the applying in step (c) is spaced out over 1, 2, or 3 applications.

In some embodiments, step (c) is carried out at resident time(s) of from or from about 1, 2, 3, 4, 5, or 6 minutes.

In some embodiments, the method further comprises, following step (e), one or more of: (f) blue dye chromatography (g) DE pad filtration step, (h) depth filtration step or (g) PS20 spiking & filtration step.

Also described herein are methods of producing a hypersialylated IgG (hsIgG) composition comprising: (a) providing hsIgG; (b) precipitating the hsIgG by adding saturated solution of ammonium sulfate, thereby producing a precipitated hsIgG solution; and (c) isolating the precipitated hsIgG, thereby producing a purified hsIgG composition

In some embodiments, isolating the precipitated hsIgG comprises filtration and/or centrifugation.

Also provided herein are methods of producing hypersialylated IgG (hsIgG) comprising: (a) providing pooled IgG antibodies; (b) incubating the pooled IgG antibodies in a reaction mixture comprising β1,4-Galactosyltransferase (B4GalT) or enzymatically active portion thereof, and UDP-Gal or salt thereof; and(c) incubating the galactosylated IgG antibodies in a reaction mixture comprising ST6Gal, or enzymatically active portion thereof, and CMP-NANA or salt thereof, (d) purifying the hsIgG according to any of the methods described herein.

Also described herein are methods of preparing hypersialylated IgG (hsIgG), comprising: (a) providing pooled IgG antibodies; (b) incubating the pooled IgG antibodies in a reaction mixture comprising β1,4-Galactosyltransferase (B4GalT) or enzymatically active portion thereof, UDP-Gal or salt thereof, ST6Gal or enzymatically active portion thereof, and CMP-NANA or salt thereof; and (c) purifying the hsIgG according to any of the methods described herein, thereby creating the hsIgG preparation.

Also described herein are methods of preparing hypersialylated IgG (hsIgG) comprising: (a) providing pooled IgG antibodies; (b) incubating the pooled IgG antibodies in a galactosylation reaction mixture comprising β1,4-Galactosyltransferase (B4GalT) or enzymatically active portion thereof, and UDP-Gal or salt thereof, thereby producing galactosylated IgG antibodies; (c) adding ST6Gal or an enzymatically active portion thereof and CMP-NANA or salt thereof to the galactosylation reaction mixture to produce a sialylation reaction mixture; (d) incubating the sialylation reaction mixture; and (e) purifying the hsIgG according to any of the methods described herein, thereby producing hsIgG

Also described herein are methods of preparing purified hypersialylated (hsIgG) composition comprising: (a) providing a mixture of IgG antibodies, (b) incubating the mixture of IgG antibodies in a reaction mixture comprising β1,4-Galactosyltransferase I (β4Ga1T) or enzymatically active portion thereof and UDP-Gal to produce galactosylated IgG antibodies; (c) incubating the galactosylated IgG antibodies in a second reaction mixture comprising ST6Gal or enzymatically active portion thereof and CMP-NANA to produce a hsIgG mixture; (d) applying the hsIgG mixture to a protein A column under conditions that bind IgG antibodies; and (e) eluting hsIgG from the protein A column.

In some embodiments, the method further comprises (f) further purifying the hsIgG produced in step (e) using a trisacryl blue column.

In some embodiments, step (e) comprises eluting the hsIgG with a buffer comprising glycine.

In some embodiments, the protein A column is washed with an acetate buffer between step (d) and step (e).

In some embodiments, the pH and salt content of hsIgG produced in step (e) is altered before applying it to the trisacryl blue column.

In some embodiments, step (f) comprises eluting the hsIgG with a high salt buffer.

In some embodiments, the high salt buffer comprises 2 M NaCl.

In some embodiments, the high salt buffer comprises 2 M KCl

In some embodiments, the hsIgG produced in step (e) is altered to 0.4 M NaCl and pH 4.5.

Also described herein are methods of preparing hypersialylated (hsIgG) comprising: (a) providing a mixture of IgG antibodies; (b) incubating the mixture of IgG antibodies in a reaction mixture comprising β1,4-Galactosyltransferase (B4GalT) or enzymatically active portion thereof, UDP-Gal, ST6Gal or enzymatically active portion thereof and CMP-NANA, thereby creating an hsIgG preparation; (d) applying the hsIgG preparation to a protein A column under conditions that bind IgG antibodies; and (e) eluting the hsIgG from the protein A column.

In some embodiments, the B4GalT or enzymatically active portion thereof is at least 90% identical SEQ ID NO: 12 or SEQ ID NO: 13 and the ST6Gal or enzymatically active portion thereof comprises an amino acid sequence that is at least 90% identical SEQ ID NO: 19 or SEQ ID NO: 20.

In some embodiments, the IgG antibodies comprise IgG antibodies isolated from at least 1000 donors.

In some embodiments, at least 70% w/w of the IgG antibodies are IgG1antibodies.

In some embodiments, at least 90% of the donor subjects have been exposed to a virus.

In some embodiments, the step of providing a mixture of IgG antibodies comprises: (a) providing pooled plasma from at least 1000 human subjects; and (b) isolating a mixture of IgG antibodies from the pooled plasma.

In some embodiments, the mixture of IgG antibodies are isolated from intravenous immunoglobulin.

In some embodiments, the mixture of IgG antibodies are intravenous immunoglobulin.

In some embodiments, the step of isolating a mixture of IgG antibodies from the pooled plasma comprises ethanol precipitation or caprylic acid precipitation.

In some embodiments, the step of isolating a mixture of IgG antibodies from the pooled plasma comprises biding IgG antibodies to an ion exchange column and eluting the IgG antibodies from an ion exchange column.

Also provided herein are methods of preparing purified hypersialylated (hsIgG) composition comprising: (a) providing a mixture of IgG antibodies, (b) incubating the mixture of IgG antibodies in a reaction mixture comprising (β1,4-Galactosyltransferase (B4GalT) or enzymatically active portion thereof and UDP-Gal to produce galactosylated IgG antibodies; (c) incubating the galactosylated IgG antibodies in a reaction mixture comprising ST6Gal or enzymatically active portion thereof and CMP-NANA to produce an hsIgG composition; and (d) isolating B4GalT or enzymatically active portion thereof and ST6Gal or enzymatically active portion thereof from the hsIgG composition.

In some embodiments, the method further comprises isolating one or both of B4GalT or enzymatically active portion thereof and ST6Gal or enzymatically active portion thereof from the hsIgG composition.

Also provided herein are methods of preparing hypersialylated (hsIgG) comprising: (a) providing a mixture of IgG antibodies; (b) incubating the mixture of IgG antibodies in a reaction mixture comprising β1,4-Galactosyltransferase (B4GalT) or enzymatically active portion thereof, UDP-Gal, ST6Gal or enzymatically active portion thereof and CMP-NANA, thereby creating an hsIgG preparation; and (c) isolating hsIgG from the hsIgG prepartion and isolating B4GalT or enzymatically active portion thereof and ST6Gal or enzymatically active portion thereof from the hsIgG preparation.

In some embodiments of any of the purification methods described herein, about 60%, 65%, 70%, 75%, 80%, or 85% of the branched glycans on the hsIgG have a sialic acid on both the α1,3 branch and the α1,6 branch before purification.

In some embodiments of any of the purification methods described herein, about 80%, or 85% of the branched Fc glycans on the hsIgG have a sialic acid on both the α1,3 branch and the α1,6 branch before purification.

In some embodiments of any of the purification methods described herein, at least 60%, 65%, 70%, 75%, 80%, or 85% of the branched glycans on the Fab domain of the hsIgG have a sialic acid on both the α 1,3 arm and the α 1,6 arm that is connected through a NeuAc-α 2,6-Gal terminal linkage before purification.

In some embodiments of any of the purification methods described herein, at least 80% of the branched Fc glycans on the hsIgG have a sialic acid on both the α1,3 branch and the α1,6 branch before purification.

In some embodiments of any of the purification methods described herein, at least 60%, 65%, 70% of the branched glycans on the Fab domain of the hsIgG have a sialic acid on both the α 1,3 arm and the α 1,6 arm that is connected through a NeuAc-α 2,6-Gal terminal linkage before purification.

In some embodiments of any of the purification methods described herein, at least 85% of the branched Fc glycans on the hsIgG have a sialic acid on both the α1,3 branch and the α1,6 branch before purification.

In some embodiments of any of the purification methods described herein, at least 60%, 65%, 70% of the branched glycans on the Fab domain of the hsIgG have a sialic acid on both the α 1,3 arm and the α 1,6 arm that is connected through a NeuAc-α 2,6-Gal terminal linkage before purification.

In some embodiments of any of the purification methods described herein, at least 90% of the branched Fc glycans on the hsIgG have a sialic acid on both the α1,3 branch and the α1,6 branch before purification.

In some embodiments of any of the purification methods described herein, at least 60%, 65%, 70% of the branched glycans on the Fab domain of the hsIgG have a sialic acid on both the α 1,3 arm and the α 1,6 arm that is connected through a NeuAc-α 2,6-Gal terminal linkage before purification.

In some embodiments of any of the purification methods described herein, about 60%, 65%, 70%, 75%, 80%, or 85% of the branched glycans on the hsIgG have a sialic acid on both the α1,3 branch and the α1,6 branch after purification.

In some embodiments of any of the purification methods described herein, about 80%, or 85% of the branched Fc glycans on the hsIgG have a sialic acid on both the α1,3 branch and the α1,6 branch after purification.

In some embodiments of any of the purification methods described herein, at least 60%, 65%, 70%, 75%, 80%, or 85% of the branched glycans on the Fab domain of the hsIgG have a sialic acid on both the α 1,3 arm and the α 1,6 arm that is connected through a NeuAc-α 2,6-Gal terminal linkage after purification.

In some embodiments of any of the purification methods described herein, at least 80% of the branched Fc glycans on the hsIgG have a sialic acid on both the α1,3 branch and the α1,6 branch after purification.

In some embodiments of any of the purification methods described herein, at least 60%, 65%, 70% of the branched glycans on the Fab domain of the hsIgG have a sialic acid on both the α 1,3 arm and the α 1,6 arm that is connected through a NeuAc-α 2,6-Gal terminal linkage after purification.

In some embodiments of any of the purification methods described herein, at least 85% of the branched Fc glycans on the hsIgG have a sialic acid on both the α1,3 branch and the α1,6 branch after purification.

In some embodiments of any of the purification methods described herein, at least 60%, 65%, 70% of the branched glycans on the Fab domain of the hsIgG have a sialic acid on both the α 1,3 arm and the α 1,6 arm that is connected through a NeuAc-α 2,6-Gal terminal linkage after purification.

In some embodiments of any of the purification methods described herein, at least 90% of the branched Fc glycans on the hsIgG have a sialic acid on both the α1,3 branch and the α1,6 branch after purification.

In some embodiments of any of the purification methods described herein, at least 60%, 65%, 70% of the branched glycans on the Fab domain of the hsIgG have a sialic acid on both the α 1,3 arm and the α 1,6 arm that is connected through a NeuAc-α 2,6-Gal terminal linkage after purification.

In some embodiments of any of the purification methods described herein, the method further comprises analyzing the amount of one or more IgG subclasses after a chromatography step.

Also described herein is a method of preparing purified hypersialylated (hsIgG) composition, the method comprising: (a) providing a mixture of IgG antibodies, (b) incubating the mixture of IgG antibodies in a reaction mixture comprising β1,4-Galactosyltransferase I (B4GalT) and UDP-Gal to produce galactosylated IgG antibodies; (c) incubating the galactosylated IgG antibodies in a second reaction mixture comprising ST6Ga11 and CMP-NANA to produce a hsIgG mixture; (d) applying the hsIgG mixture to a protein A column under conditions that bind IgG antibodies; and (e) eluting hsIgG from the protein A column.

In various embodiments the method further comprises: (g) further purifying the hsIgG produced in step (e) using a trisacryl blue column; the B4GalT is at least 90% identical SEQ ID NO:1 or SEQ ID NO:2; the ST6Ga11 comprises an amino acid sequence that is at least 90% identical SEQ ID NO:3 or SEQ ID NO:4 or SEQ ID NO:5; step (e) comprises eluting the hsIgG with a buffer comprising glycine; the protein A column is washed with an acetate buffer between step (d) and step (e); the pH and salt content of hsIgG produced in step (e) is altered before applying it to the trisacryl blue column; step (g) comprises eluting the hsIgG with a high salt buffer; the high salt buffer comprises 2 M NaCl; the high salt buffer comprises 2 M KCl; and/or the hsIgG produced in step (e) is altered to 0.4 M NaCl and pH 4.5.

Also described herein is method of preparing hypersialylated (hsIgG), the method comprising (a) providing a mixture of IgG antibodies; (b) incubating the mixture of IgG antibodies in a reaction mixture comprising β1,4-Galactosyltransferase I (B4GalT), UDP-Gal, ST6Gal1 and CMP-NANA, thereby creating an hsIgG preparation; (d) applying the hsIgG composition to a protein A column under conditions that bind IgG antibodies; and (e) eluting the IgG antibodies from the protein A column.

Also described is a method of preparing purified hypersialylated (hsIgG) composition, the method comprising (a) providing a mixture of IgG antibodies, (b) incubating the mixture of IgG antibodies in a reaction mixture comprising β1,4-Galactosyltransferase I (B4GalT) and UDP-Gal to produce galactosylated IgG antibodies; (c) incubating the galactosylated IgG antibodies in a reaction mixture comprising ST6Ga11 and CMP-NANA to produce an hsIgG composition; and (d) isolating hsIgG from the hsIgG composition and isolating B4GalT and ST6Ga11 from the hsIgG composition.

Also described is a method of preparing hypersialylated (hsIgG), the method comprising (a) providing a mixture of IgG antibodies; (b) incubating the mixture of IgG antibodies in a reaction mixture comprising β1,4-Galactosyltransferase I (B4GalT), UDP-Gal, ST6Gal1 and CMP-NANA, thereby creating an hsIgG preparation; and (c) isolating hsIgG from the hsIgG composition and isolating B4GalT and ST6Gal1 from the hsIgG composition.

In various embodiments of the all of the forgoing methods: the B4GalT is at least 90% identical SEQ ID NO: 12 or SEQ ID NO: 13 and the ST6Ga11 comprises an amino acid sequence that is at least 90% identical SEQ ID NO: 19 or SEQ ID NO: 20; the IgG antibodies comprise IgG antibodies isolated from at least 1000 donors; at least 70% w/w of the IgG antibodies are IgG1antibodies; at least 90% of the donor subjects have been exposed to a virus; at least 60%, 65%, 70%, 75%, 80%, or 85% of the branched glycans on the IgG antibodies in the hsIgG preparation have a sialic acid on both the α1,3 branch and the α1,6 branch; at least 60%, 65%, 70%, 75%, 80%, or 85% of the branched glycans on the Fab domain of the IgG antibodies in the hsIgG preparation have a sialic acid on both the α 1,3 arm and the α 1,6 arm; the step of providing a mixture of IgG antibodies comprises: (a) providing pooled plasma from at least 1000 human subjects; and (b) isolating a mixture of IgG antibodies from the pooled plasma; the mixture of IgG antibodies are isolated from intravenous immunoglobulin; the mixture of IgG antibodies are intravenous immunoglobulin; the step of isolating a mixture of IgG antibodies from the pooled plasma comprises ethanol precipitation or caprylic acid precipitation; and the step of isolating a mixture of IgG antibodies from the pooled plasma comprises biding IgG antibodies to an ion exchange column and eluting the IgG antibodies from an ion exchange column.

In hypersialylated IgG at least 60% (e.g., 65%, 70%, 75%, 80%, 82%, 85%, 87%, 90%, 92%, 94%, 95%, 97%, 98% up to and including 100%) of branched glycans on the Fc region are di-sialylated (i.e., on both the α 1,3 branch and the α 1,6 arm) by way of NeuAc-α 2,6-Gal terminal linkages. In some embodiments, less than 50% (e.g., less than 40%, 30%, 20%, 15%, 10%, 5%, 4%, 3%, 2%, 1%) of branched glycans on the Fc region are mono-sialylated (i.e., sialylated only on the α 1,3 branch or only on the α 1,6 branch) by way of a NeuAc-α 2,6-Gal terminal linkage.

In some embodiments, the polypeptides are derived from plasma, e.g., human plasma. In certain embodiments, the polypeptides are overwhelmingly IgG polypeptides (e.g., IgG1, IgG2, IgG3 or IgG4 or mixtures thereof), although trace amounts of other of other immunoglobulin subclasses can be present.

As used herein, the term “antibody” refers to a polypeptide that includes at least one immunoglobulin variable region, e.g., an amino acid sequence that provides an immunoglobulin variable domain or immunoglobulin variable domain sequence. For example, an antibody can include a heavy (H) chain variable region (abbreviated herein as V_{H}), and a light (L) chain variable region (abbreviated herein as V_{L}). In another example, an antibody includes two heavy (H) chain variable regions and two light (L) chain variable regions. The term “antibody” encompasses antigen-binding fragments of antibodies (e.g., single chain antibodies, Fab, F(ab′)₂, Fd, Fv, and dAb fragments) as well as complete antibodies, e.g., intact immunoglobulins of types IgA, IgG, IgE, IgD, IgM (as well as subtypes thereof). The light chains of the immunoglobulin can be of types kappa or lambda.

As used herein, the term “constant region” refers to a polypeptide that corresponds to, or is derived from, one or more constant region immunoglobulin domains of an antibody. A constant region can include any or all of the following immunoglobulin domains: a C_{H}1 domain, a hinge region, a C_{H}2 domain, a C_{H}3 domain (derived from an IgA, IgD, IgG, IgE, or IgM), and a C_{H}4 domain (derived from an IgE or IgM).

As used herein, the term “Fc region” refers to a dimer of two “Fc polypeptides,” each “Fc polypeptide” including the constant region of an antibody excluding the first constant region immunoglobulin domain. In some embodiments, an “Fc region” includes two Fc polypeptides linked by one or more disulfide bonds, chemical linkers, or peptide linkers. “Fc polypeptide” refers to the last two constant region immunoglobulin domains of IgA, IgD, and IgG, and the last three constant region immunoglobulin domains of IgE and IgM, and may also include part or the entire flexible hinge N-terminal to these domains. For IgG, “Fc polypeptide” comprises immunoglobulin domains Cgamma2 (Cγ2) and Cgamma3 (Cγ3) and the lower part of the hinge between Cgamma1 (Cγl) and Cγ2. Although the boundaries of the Fc polypeptide may vary, the human IgG heavy chain Fc polypeptide is usually defined to comprise residues starting P232, to its carboxyl-terminus, wherein the numbering is according to the EU system (Edelman et al., Proc. Natl. Acad. USA, 63, 78-85 (1969)). For IgA, Fc polypeptide comprises immunoglobulin domains Calpha2 (Cα2) and Calpha3 (Cα3) and the lower part of the hinge between Calphal (Cα1) and Cα2. An Fc region can be synthetic, recombinant, or generated from natural sources such as IVIg.

As used herein, “glycan” is a sugar, which can be monomers or polymers of sugar residues, such as at least three sugars, and can be linear or branched. A “glycan” can include natural sugar residues (e.g., glucose, N-acetylglucosamine, N-acetyl neuraminic acid, galactose, mannose, fucose, hexose, arabinose, ribose, xylose, etc.) and/or modified sugars (e.g., 2′-fluororibose, 2′-deoxyribose, phosphomannose, 6′sulfo N-acetylglucosamine, etc.). The term “glycan” includes homo and heteropolymers of sugar residues. The term “glycan” also encompasses a glycan component of a glycoconjugate (e.g., of a polypeptide, glycolipid, proteoglycan, etc.). The term also encompasses free glycans, including glycans that have been cleaved or otherwise released from a glycoconjugate.

As used herein, the term “glycoprotein” refers to a protein that contains a peptide backbone covalently linked to one or more sugar moieties (i.e., glycans). The sugar moiety(ies) may be in the form of monosaccharides, disaccharides, oligosaccharides, and/or polysaccharides. The sugar moiety(ies) may comprise a single unbranched chain of sugar residues or may comprise one or more branched chains. Glycoproteins can contain O-linked sugar moieties and/or N-linked sugar moieties.

As used herein, “IVIg” is a preparation of pooled, polyvalent IgG, including all four IgG subgroups, extracted from plasma of at least 1,000 human donors. IVIg is approved as a plasma protein replacement therapy for immune deficient patients. The level of IVIg Fc glycan sialylation varies among IVIg preparations, but is generally less than 20%. The level of disialylation is generally far lower than 20%. As used herein, the term “derived from IVIg” refers to polypeptides which result from manipulation of IVIg. For example, polypeptides purified from IVIg (e.g., enriched for sialylated IgGs or modified IVIg (e.g., IVIg IgGs enzymatically sialylated).

As used herein, an “N-glycosylation site of an Fc polypeptide” refers to an amino acid residue within an Fc polypeptide to which a glycan is N-linked. In some embodiments, an Fc region contains a dimer of Fc polypeptides, and the Fc region comprises two N-glycosylation sites, one on each Fc polypeptide.

As used herein “percent (%) of branched glycans” refers to the number of moles of glycan X relative to total moles of glycans present, wherein X represents the glycan of interest.

The term “pharmaceutically effective amount” or “therapeutically effective amount” refers to an amount (e.g., dose) effective in treating a patient, having a disorder or condition described herein. It is also to be understood herein that a “pharmaceutically effective amount” may be interpreted as an amount giving a desired therapeutic effect, either taken in one dose or in any dosage or route, taken alone or in combination with other therapeutic agents.

“Pharmaceutical preparations” and “pharmaceutical products” can be included in kits containing the preparation or product and instructions for use.

“Pharmaceutical preparations” and “pharmaceutical products” generally refer to compositions in which the final predetermined level of sialylation has been achieved, and which are free of process impurities. To that end, “pharmaceutical preparations” and “pharmaceutical products” are substantially free of ST6Gal1 and/or sialic acid donor (e.g., cytidine 5′-monophospho-N-acetyl neuraminic acid) or the byproducts thereof (e.g., cytidine 5′-monophosphate).

“Pharmaceutical preparations” and “pharmaceutical products” are generally substantially free of other components of a cell in which the glycoproteins were produced (e.g., the endoplasmic reticulum or cytoplasmic proteins and RNA), if recombinant.

By “purified” (or “isolated”) refers to a polynucleotide or a polypeptide that is removed or separated from other components present in its natural environment. For example, an isolated polypeptide is one that is separated from other components of a cell in which it was produced (e.g., the endoplasmic reticulum or cytoplasmic proteins and RNA). An isolated polynucleotide is one that is separated from other nuclear components (e.g., histones) and/or from upstream or downstream nucleic acids. An isolated polynucleotide or polypeptide can be at least 60% free, or at least 75% free, or at least 90% free, or at least 95% free from other components present in natural environment of the indicated polynucleotide or polypeptide.

As used herein, the term “sialylated” refers to a glycan having a terminal sialic acid. The term “mono-sialylated” refers to branched glycans having one terminal sialic acid, e.g., on an α1,3 branch or an α1,6 branch. The term “di-sialylated” refers to a branched glycan having a terminal sialic acid on two arms, e.g., both an α1,3 arm and an α1,6 arm.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 shows a short, branched core oligosaccharide comprising two N-acetylglucosamine and three mannose residues. One of the branches is referred to in the art as the “α 1,3 arm,” and the second branch is referred to as the “α 1,6 arm,”. Squares: N-acetylglucosamine; dark gray circles: mannose; light gray circles: galactose; diamonds: N-acetylneuraminic acid; triangles: fucose.

FIG. 2 shows common Fc glycans present in IVIg. Squares: N-acetylglucosamine; dark gray circles: mannose; light gray circles: galactose; diamonds: N-acetylneuraminic acid; triangles: fucose.

FIG. 3 shows how immunoglobulins, e.g., IgG antibodies, can be sialylated by carrying out a galactosylation step followed by a sialylation step. Squares: N-acetylglucosamine; dark gray circles: mannose; light gray circles: galactose; diamonds: N-acetylneuraminic acid; triangles: fucose.

FIG. 4 shows the reaction product of a representative example of the IgG-Fc glycan profile for a reaction starting with IVIg. The left panel is a schematic representation of enzymatic sialylation reaction to transform IgG to hsIgG; the right panel is the IgG Fc glycan profile for the starting IVIg and hsIgG. Bars, from left to right, correspond to IgGl, IgG2/3, and IgG3/4, respectively.

FIG. 5 shows an exemplary production process for hsIgG.

FIG. 6 shows binding capacity of the Poros™ resin.

FIG. 7 shows a prediction profiler for yield and residual ST6.

FIG. 8 shows the operating range for a yield between 90- 95% and residual ST6 levels less than 30 ppm.

FIG. 9 is an SEC chromatogram of flow through.

FIG. 10 is an SEC of eluate.

FIG. 11 is an overlay of TAB, Capto™ blue and Capto™ Blue Sepharose 2 FF.

FIG. 12 shows recovery of the three runs (bars, from left to rightTAB, Capto™ Blue and Blue Sepharose™ FF) showing similar recovery.

FIG. 13 shows chromatograms overlay of run 1 and 4.

FIG. 14 shows residual ST6 of the 4 runs that demonstrates a better clearance by Capto™ Blue HS (runs 3 and 4) versus Capto™ Blue (Runs 1 and 2).

FIG. 15 shows a comparison of residual ST6 of Capto™ Blue HS and TAB showing a better clearance of the Capto™ Blue HS.

FIG. 16 shows analytical SEC of the IVIg load, FT, and eluate.

FIG. 17 shows mass Spectrometry of Capto™ Blue HS IVIg flow through and eluate against the starting IVIg. Bars, from left to right: IgG1, IgG2, IgG3, IgG4.

FIG. 18 shows the impact of 5X PBS and MOPS at pH 4.5 buffer on residual ST6 (run 1). X axis: mg IVIg/mL resin.

FIG. 19 shows the impact of Glycine pH 4.5 on residual ST6 (Run2). X axis: mg IVIg/mL resin.

FIG. 20 shows the impact of Glycine +100 mM NaCl at pH 4.5 on residual ST6 (Run2).

FIG. 21 shows the amount of loading (grams of M254/ml column volume) impact on the clearance of ST6 by TAB Column. Orange and blue dots represent residual level of ST6 for the TAB column flow-through fractions at different loading amounts of CEX load and CEX eluate respectively. Achieved similar level of residual ST6 for the TAB column flow-through fractions with both the loads (CEX load and CEX eluate) at current manufacturing scale maximum loading 1.1 g/ml (1X).

FIG. 22 is an overlay of the analytical SEC of IVIg and precipitated IVIg showing no change on the aggregate level.

FIG. 23 is an analytical SEC profile of the crude reaction versus the precipitated material showing a significant reduction of nucleotides.

### DETAILED DESCRIPTION

Antibodies are glycosylated at conserved positions in the constant regions of their heavy chain and on the Fab domain. For example, human IgG antibodies have a single N-linked glycosylation site at Asn297 (EU Numbering) of the CH2 domain. Each antibody isotype has a distinct variety of N-linked carbohydrate structures in the constant regions. For human IgG, the core oligosaccharide normally consists of G1cNAc₂Man₃G1cNAc, with differing numbers of outer residues. Variation among individual IgG’s can occur via attachment of galactose and/or galactose-sialic acid at one or both terminal GlcNAc or via attachment of a third GlcNAc arm (bisecting GlcNAc).

The present disclosure encompasses, in part, methods for preparing immunoglobulins (e.g., human IgG) having an Fc region having particular levels of branched glycans that are sialylated on both of the arms of the branched glycan (e.g., with a NeuAc-α 2,6-Gal terminal linkage). The levels can be measured on an individual Fc region (e.g., the number of branched glycans that are sialylated on an α1,3 arm, an α1,6 arm, or both, of the branched glycans in the Fc region), or on the overall composition of a preparation of polypeptides (e.g., the number or percentage of branched glycans that are sialylated on an α1,3 arm, an α1,6 arm, or both, of the branched glycans in the Fc region in a preparation of polypeptides).

Naturally derived polypeptides that can be used to prepare hypersialylated IgG include, for example, IgG in human serum (particular human serum pooled from more than 1,000 donors), intravenous immunoglobulin (IVIg) and polypeptides derived from IVIg (e.g., polypeptides purified from IVIg (e.g., enriched for sialylated IgGs) or modified IVIg (e.g., IVIg IgGs enzymatically sialylated).

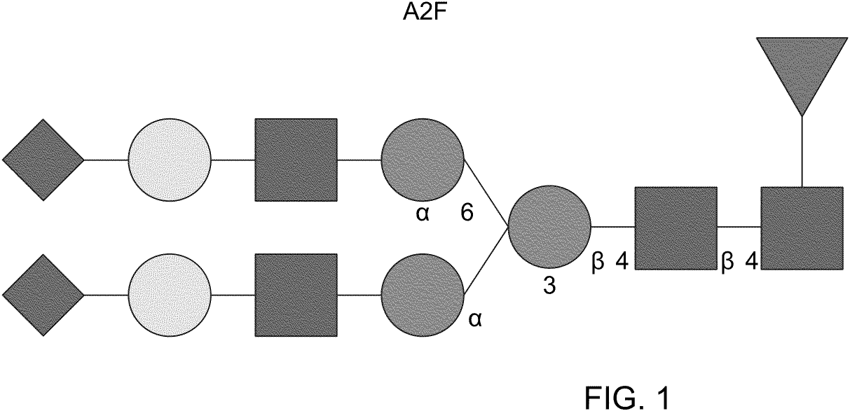

N-linked oligosaccharide chains are added to a protein in the lumen of the endoplasmic reticulum. Specifically, an initial oligosaccharide (typically 14-sugar) is added to the amino group on the side chain of an asparagine residue contained within the target consensus sequence of Asn-X-Ser/Thr, where X may be any amino acid except proline. The structure of this initial oligosaccharide is common to most eukaryotes, and contains three glucose, nine mannose, and two N-acetylglucosamine residues. This initial oligosaccharide chain can be trimmed by specific glycosidase enzymes in the endoplasmic reticulum, resulting in a short, branched core oligosaccharide composed of two N-acetylglucosamine and three mannose residues. One of the branches is referred to in the art as the “α 1,3 arm,” and the second branch is referred to as the “α 1,6 arm,” as shown in FIG. 1.

N-glycans can be subdivided into three distinct groups called “high mannose type,” “hybrid type,” and “complex type,” with a common pentasaccharide core (Man (α 1,6)-(Man(α 1,3))-Man((β 1,4)-GlcpNAc((β 1,4)-GlcpNAc(β 1,N)-Asn) occurring in all three groups.

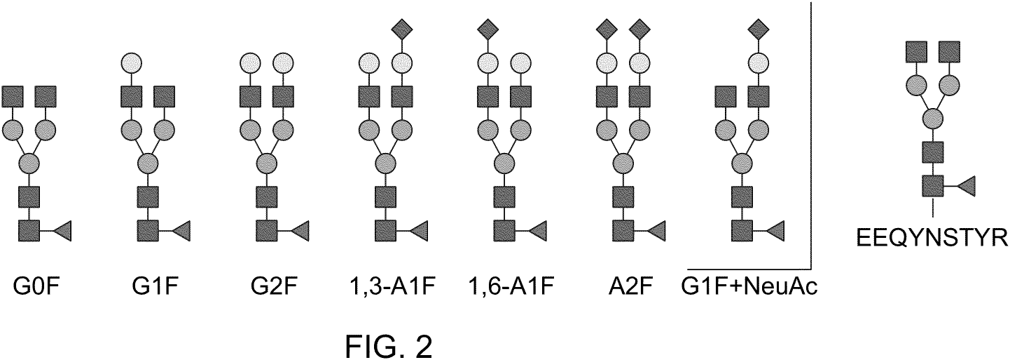

The more common Fc glycans present in IVIg are shown in FIG. 2.

Additionally or alternatively, one or more monosaccharides units of N-acetylglucosamine may be added to the core mannose subunits to form a “complex glycan.” Galactose may be added to the N-acetylglucosamine subunits, and sialic acid subunits may be added to the galactose subunits, resulting in chains that terminate with any of a sialic acid, a galactose or an N-acetylglucosamine residue. Additionally, a fucose residue may be added to an N-acetylglucosamine residue of the core oligosaccharide. Each of these additions is catalyzed by specific glycosyl transferases.

“Hybrid glycans” comprise characteristics of both high-mannose and complex glycans. For example, one branch of a hybrid glycan may comprise primarily or exclusively mannose residues, while another branch may comprise N-acetylglucosamine, sialic acid, galactose, and/or fucose sugars.

Sialic acids are a family of 9-carbon monosaccharides with heterocyclic ring structures. They bear a negative charge via a carboxylic acid group attached to the ring as well as other chemical decorations including N-acetyl and N-glycolyl groups. The two main types of sialic acid residues found in polypeptides produced in mammalian expression systems are N-acetyl-neuraminic acid (NeuAc) and N-glycolylneuraminic acid (NeuGc). These usually occur as terminal structures attached to galactose (Gal) residues at the non-reducing termini of both N-and O-linked glycans. The glycosidic linkage configurations for these sialic acid groups can be either α 2,3 or α 2,6.

Fc regions are glycosylated at conserved, N-linked glycosylation sites. For example, each heavy chain of an IgG antibody has a single N-linked glycosylation site at Asn297 of the CH2 domain. IgA antibodies have N-linked glycosylation sites within the CH2 and CH3 domains, IgE antibodies have N-linked glycosylation sites within the CH3 domain, and IgM antibodies have N-linked glycosylation sites within the CH1, CH2, CH3, and CH4 domains.

Each antibody isotype has a distinct variety of N-linked carbohydrate structures in the constant regions. For example, IgG has a single N-linked biantennary carbohydrate at Asn297 of the CH2 domain in each Fc polypeptide of the Fc region, which also contains the binding sites for C1q and FcγR. For human IgG, the core oligosaccharide normally consists of GlcNAc2Man3GlcNAc, with differing numbers of outer residues. Variation among individual IgG can occur via attachment of galactose and/or galactose-sialic acid at one or both terminal GlcNAc or via attachment of a third GlcNAc arm (bisecting GlcNAc).

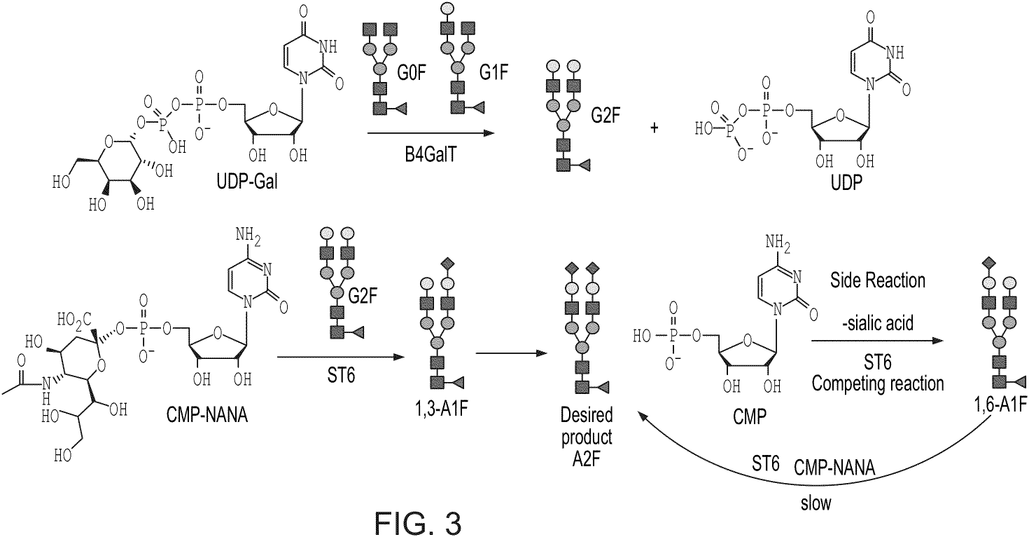

Immunoglobulins, e.g., IgG antibodies, can be sialylated by carrying out a galactosylation step followed by a sialylation step. Beta-1,4-galactosyltransferase 1 (B4GalT) is a Type II Golgi membrane-bound glycoprotein that transfers galactose from uridine 5′-diphosphosegalactose ([[(2R,3S,4R,SR)-5-(2,4-dioxopyrimidin-1-yl)-3,4-dihydroxyoxolan-2-yl]methoxy-hydroxyphosphoryl] [(2R,3R,4S,5R,6R)-3,4,5-trihydroxy-6-(hydroxymethyl)oxan-2-yl] hydrogen phosphate; UDP-Gal) to GlcNAc as a β-1,4 linkage. Alpha-2,6-sialyltransferase 1 (ST6) is a Type II Golgi membrane-bound glycoprotein that transfers sialic acid from cytidine 5′-monophospho-N-acetylneuraminicacid ((2R,4S,5R,6R)-5-acetamido-2-[[(2R,3S,4R,5R)-5-(4-amino-2-oxopyrimidin-1-yl)-3,4-dihydroxyoxolan-2-yl]methoxy-hydroxyphosphoryl]oxy-4-hydroxy-6-(1,2,3-trihydroxypropyl)oxane-2-carboxylic acid; CMP-NANA or CMP-Sialic Acid) to Gal as an α-2,6 linkage. Schematically, the reactions proceed shown in FIG. 3.

Glycans of polypeptides can be evaluated using any methods known in the art. For example, sialylation of glycan compositions (e.g., level of branched glycans that are sialylated on an α1,3 branch and/or an α1,6 branch) can be characterized using methods described in WO2014/179601.

In some embodiments of the hsIgG compositions prepared by the methods described herein, at least 60%, 65%, 70%, 75%, 80%, 85%, or 90% of the branched glycans on the Fc domain have a sialic acid on both the α 1,3 arm and the α 1,6 arm that is connected through a NeuAc-α 2,6-Gal terminal linkage. In addition, in some embodiments, at least 40%, 50%, 60%, 65%, 70%, 75%, 80%, or 85% of the branched glycans on the Fab domain have a sialic acid on both the α 1,3 arm and the α 1,6 arm that is connected through a NeuAc-α 2,6-Gal terminal linkage. Overall, in some embodiments, at least 60%, 65%, 70%, 75%, 80%, 85%, or 90% of the branched glycans have a sialic acid on both the α 1,3 arm and the α 1,6 arm that is connected through a NeuAc-α 2,6-Gal terminal linkage.

In some embodiments, the hsIgG compositions prepared by the methods described herein comprises at least 50%, 55%, 60%, 65%, 70% or 75% of the branched glycans on the Fc domain have a sialic acid on both the α 1,3 arm and the α 1,6 arm.

### Enzymes

### Galactosylating Enzymes

Beta-1,4-galactosyltransferase (B4GalT), e.g., human B4GalT, e.g., human (B4Galt1), as well as orthologs, mutants, and variants thereof, including enzymatically active portions of beta-1,4-galactosyltransferase (B4GalT), e.g., human B4GalT, e.g., human B4Galt1, as well as orthologs, mutants, and variants thereof, along with fusion proteins and polypeptides comprising the same are suitable for use in the methods described herein. B4Galt1 is one of seven beta-1,4-galactosyltransferase (beta4GalT) genes that each encode type II membrane-bound glycoproteins that appear to have exclusive specificity for the donor substrate UDP-galactose; all transfer galactose in a beta 1,4 linkage to similar acceptor sugars: GlcNAc, Glc, and Xyl. B4Galt1 adds galactose to N-acetylglucosamine residues that are either monosaccharides or the nonreducing ends of glycoprotein carbohydrate chains. B4GalT1 is also called GGTB2. Four alternative transcripts encoding four isoforms of B4GALT1 (NCBI Gene ID 2683) are described in Table 1.

TABLE 1

Human B4GALT1 isoforms

| Transcript | Length (nt) | Protein | SEQ ID NO: | Length (aa) | Isoform |
|---|---|---|---|---|---|
| NM_001497.4 | 4176 | NP_001488.2 | SEQ ID NO: 5 | 398 | 1 |
| NM_001378495.1 | 3999 | NP_001365424.1 | SEQ ID NO: 6 | 385 | 2 |
| NM_001378496.1 | 4053 | NP_001365425.1 | SEQ ID NO: 7 | 357 | 3 |
| NM_001378497.1 | 1520 | NP_001365426.1 | SEQ ID NO: 8 | 225 | 4 |

TABLE 2

Topology of B4GALT1 isoform 1 (SEQ ID NO: 5)

| Feature | AAs | Description | Length | Sequence | SEQ ID NO: |
|---|---|---|---|---|---|
| Topological domain | 1-24 | Cytoplasmic | 9 | MRLREPLLSGSAAMPGASLQRACR | SEQ ID NO: 9 |
| Transmembr ane | 25 – 44 | Helical; Signal-anchor for type II membrane protein | 17 | LLVAVCALHLGVTLVYYLAG | SEQ ID NO: 10 |
| Topological domain | 45 – 398 | Lumenal | 380 | RDLSRLPQLVGVSTPLQGGSNSAAAIGQSSGELRTGGARPPPPLGASSQPRPGGDSSPWDSGPGPASNLTSVPVPHTTALSLPACPEESPLLVGPMLIEFNMPVDLELVAKQNPNVKMGGRYAPRDCVSPHKVAIIIPFRNRQEHLKYWLYYLHPVLQRQQLDYGIYVINQAGDTIFNRAKLLNVGFQEALKDYDYTCFVFSDVDLIPMNDHNAYRCFSQPRHISVAMDKFGFSLPYVQYFGGVSALSKQQFLTINGFPNNYWGWGGEDDDIFNRLVFRGMSISRPNAVVGRCRMIRHSRDKKNEPNPQRFDRIAHTKETMLSDGLNSLTYQVLDVQRYPLYTQITVDIGTPS | SEQ ID NO: 11 |

TABLE 3

Binding sites of B4GALT1 isoform 1 (SEQ ID NO: 5)

| Position(s) | Description | Reference(s) |
|---|---|---|
| 250 | Metal binding; Manganese | |
| 310 | Binding site; UDP-alpha-D-galactose | “Structural snapshots of beta-1,4-galactosyltransferase-I along the kinetic pathway.” Ramakrishnan B., Ramasamy V., Qasba P.K. J. Mol. Biol. 357:1619-1633(2006) |
| 343 | Metal binding; Manganese; via tele nitrogen | |
| 355 | Binding site; N-acetyl-D-glucosamine | “Oligosaccharide preferences of beta1,4-galactosyltransferase-I: crystal structures of Met340His mutant of human beta1,4-galactosyltransferase-I with a pentasaccharide and trisaccharides of the N-glycan moiety.” Ramasamy V., Ramakrishnan B., Boeggeman E., Ratner D.M., Seeberger P.H., Qasba P.K. J. Mol. Biol. 353:53-67(2005) “Deoxygenated disaccharide analogs as specific inhibitors of beta1-4-galactosyltransferase 1 and selectin-mediated tumor metastasis.” Brown J.R., Yang F., Sinha A., Ramakrishnan B., Tor Y., Qasba P.K., Esko J.D. J. Biol. Chem. 284:4952-4959(2009) |

TABLE 4

Post Translational Amino Acid Modifications of B4GALT1 isoform 1 (SEQ ID NO: 5)

| Feature key | Position(s) | Description | Reference(s) |
|---|---|---|---|
| Glycosylation | 113 | N-linked (GlcNAc...) asparagine | |
| Disulfide bond | 130 ↔ 172 | | “Oligosaccharide preferences of beta1,4-galactosyltransferase-I: crystal structures of Met340His mutant of human beta1,4-galactosyltransferase-I with a pentasaccharide and trisaccharides of the N-glycan moiety.” Ramasamy V., Ramakrishnan B., Boeggeman E., Ratner D.M., Seeberger P.H., Qasba P.K. J. Mol. Biol. 353:53-67(2005) “Structural snapshots of beta-1,4-galactosyltransferase-I along the kinetic pathway.” Ramakrishnan B., Ramasamy V., Qasba P.K. J. Mol. Biol. 357:1619-1633(2006) |
| Disulfide bond | 243 ↔ 262 | | |

The soluble form of B4GalT1 derives from the membrane form by proteolytic processing. The cleavage site is at positions 77-78 of B4GALT1 isoform 1 (SEQ ID NO: 5).

In some embodiments, one or more of the amino acids of the B4GalT1 corresponding to amino acids 113, 130, 172, 243, 250, 262, 310, 343, or 355 of B4GALT1 isoform 1 (SEQ ID NO: 5) is conserved as compared to (SEQ ID NO: 5).

In some embodiments, the enzyme is an enzymatically active portion of, e.g., B4GalT1. In some embodiments, the enzyme is an enzymatically active portion of B4GALT1 isoform 1 (SEQ ID NO: 5), or an ortholog, mutant, or variant of SEQ ID NO: 5. In some embodiments, the enzyme is an enzymatically active portion of B4GALT1 isoform 2 (SEQ ID NO: 6), or an ortholog, mutant, or variant of SEQ ID NO: 6. In some embodiments, the enzyme is an enzymatically active portion of B4GALT1 isoform 3 (SEQ ID NO: 7), or an ortholog, mutant, or variant of SEQ ID NO: 7. In some embodiments, the enzyme is an enzymatically active portion of B4GALT1 isoform 4 (SEQ ID NO: 8), or an ortholog, mutant, or variant of SEQ ID NO: 8.

In some embodiments, the enzymatically active portion of B4GalT1 does not comprise a cytoplasmic domain, e.g., SEQ ID NO: 9. In some embodiments, the enzymatically active portion of B4GalT1 does not comprise a transmembrane domain, e.g., SEQ ID NO: 10. In some embodiments, the enzymatically active portion of B4GalT1 does not comprise a cytoplasmic domain, e.g., SEQ ID NO: 9 or a transmembrane domain, e.g., SEQ ID NO: 10.

In some embodiments, the enzymatically active portion of B4GalT1 comprises all or a portion of a luminal domain, e.g., SEQ ID NO: 11, or an ortholog, mutants, or variants thereof.

In some embodiments, the enzymatically active portion of B4GalT1 comprises amino acids 109-398 of SEQ ID NO: 5, or an ortholog, mutants, or variants thereof. In some embodiments, the enzymatically active portion of B4GalT1 consists of SEQ ID NO: 5, or an ortholog, mutant, or variant of SEQ ID NO: 5.

A suitable functional portion of an B4GalT1 can comprise or consist of an amino acid sequence that is at least 80% (85%, 90%, 95%, 98% or 100%) identical to SEQ ID NO: 12.

Also suitable for use in the methods described herein is an amino acid sequence that comprises or consists of an amino acid sequence that is at least 80% (85%, 90%, 95%, 98% or 100%) identical to SEQ ID NO: 13.

### Sialylating Enzymes

ST6, e.g., ST6Ga11, e.g., human ST6Ga11, as well as orthologs, mutants, and variants thereof, including enzymatically active portions of ST6Ga11, e.g., human ST6Ga11, as well as orthologs, mutants, and variants thereof, along with fusion proteins and polypeptides comprising the same, are suitable for use in the methods described herein. Alpha-2,6-sialyltransferase 1 (ST6) is a Type II Golgi membrane-bound glycoprotein that transfers sialic acid from cytidine 5′-monophospho-N-acetylneuraminic acid (CMP-NANA) to Gal as an α-2,6 linkage. ST6Gal1 is also called as ST6N or SIAT1. Four alternative transcripts encoding two isoforms of ST6GAL1 (NCBI Gene ID 6480) are described in Table 5.

TABLE 5

Human ST6GAL1 isoforms

| Transcript | Length (nt) | Protein | SEQ ID NO: | Length (aa) | Isoform |
|---|---|---|---|---|---|
| NM_173216.2 | 4604 | NP_775323.1 | SEQ ID NO: 14 | 406 | a |
| NM_173217.2 | 3947 | NP_775324.1 | SEQ ID NO: 15 | 175 | b |
| NM_003032.3 | 4303 | NP_003023.1 | SEQ ID NO: 14 | 406 | a |
| NM_001353916.2 | 4177 | NP_001340845.1 | SEQ ID NO: 14 | 406 | a |

TABLE 6

Topology of ST6Ga11 isoform a (SEQ ID NO: 14)

| Feature | AAs | Description | Length | Sequence | SEQ ID NO: |
|---|---|---|---|---|---|
| Topological domain | 1-9 | Cytoplasmic | 9 | MIHTNLKKK | SEQ ID NO: 16 |
| Transmembrane | 10 – 26 | Helical; Signal-anchor for type II membrane protein | 17 | FSCCVLVFLLFAVICVW | SEQ ID NO: 17 |
| Topological domain | 27 – 406 | Lumenal | 380 | KEKKKGSYYDSFKLQTKEFQVLKSLG KLAMGSDSQSVSSSSTQDPHRGRQTLGSLRGLAKAKPEASFQVWNKDSSSKNLIPRLQKIWKNYLSMNKYKVSYKGPGPGIKFSAEALRCHLRDHVNVSMVEVTDFPFNTSEWEGYLPKESIRTKAGPWGRCAVVSSAGSLKSSQLGREIDDHDAVLRFNGAPTANFQQDVGTKTTIRLMNSQLVTTEKRFLKDSLYNEGILIVWDPSVYHSDIPKWYQNPDYNFFNNYKTYRKLHPNQPFYILKPQMPWELWDILQEISPEEIQPNPPSSGMLGIIIMMTLCDQVDIYEFLPSKRKTDVCYYYQKFFDSACTMGAYHPLLYEKNLVKHLNQGTDEDIYLLGKATLPGFRTIHC | SEQ ID NO: 18 |

TABLE 7

Binding sites of ST6Gal1 isoform a (SEQ ID NO: 14)

| Position(s) | Description | Reference(s) |
|---|---|---|
| 189 | Substrate; via amide nitrogen | “The structure of human alpha-2,6-sialyltransferase reveals the binding mode of complex glycans.” Kuhn B., Benz J., Greif M., Engel A.M., Sobek H., Rudolph M.G. Acta Crystallogr. D 69:1826-1838(2013) |
| 212 | Substrate | |
| 233 | Substrate | |
| 353 | Substrate; via carbonyl oxygen | |
| 354 | Substrate | |
| 365 | Substrate | |
| 369 | Substrate | |
| 370 | Substrate | “The structure of human alpha-2,6-sialyltransferase reveals the binding mode of complex glycans.” Kuhn B., Benz J., Greif M., Engel A.M., Sobek H., Rudolph M.G. Acta Crystallogr. D 69:1826-1838(2013) |
| 376 | Substrate | |

TABLE 8

Post Translational Amino Acid Modifications of ST6Gal1 isoform a (SEQ ID NO: 14)

| Feature key | Position(s) | Description | Reference(s) |
|---|---|---|---|
| Disulfide bond | 142 ↔ 406 | | “The structure of human alpha-2,6-sialyltransferase reveals the binding mode of complex glycans.” Kuhn B., Benz J., GreifM., Engel A.M., Sobek H., Rudolph M.G. Acta Crystallogr. D 69:1826-1838(2013) |
| Glycosylation | 149 | N-linked (GlcNAc...) asparagine | “Glycoproteomics analysis of human liver tissue by combination of multiple enzyme digestion and hydrazide chemistry.” Chen R., Jiang X., Sun D., Han G., Wang F., Ye M., Wang L., Zou H. J. Proteome Res. 8:651-661(2009); and “The structure of human alpha-2,6-sialyltransferase reveals the binding mode of complex glycans.” Kuhn B., Benz J., GreifM., Engel A.M., Sobek H., Rudolph M.G. Acta Crystallogr. D 69:1826-1838(2013) |
| Glycosylation | 161 | N-linked (G1cNAc...) asparagine | “Glycoproteomics analysis of human liver tissue by combination of multiple enzyme digestion and hydrazide chemistry.” Chen R., Jiang X., Sun D., Han G., Wang F., Ye M., Wang L., Zou H. J. Proteome Res. 8:651-661(2009) |
| Disulfide bond | 184 ↔ 335 | | “The structure of human alpha-2,6-sialyltransferase reveals the binding mode of complex glycans.” Kuhn B., Benz J., GreifM., Engel A.M., Sobek H., Rudolph M.G. Acta Crystallogr. D 69:1826-1838(2013) |
| Disulfide bond | 353 ↔ 364 | | “The structure of human alpha-2,6-sialyltransferase reveals the binding mode of complex glycans.” Kuhn B., Benz J., GreifM., Engel A.M., Sobek H., Rudolph M.G. Acta Crystallogr. D 69:1826-1838(2013) |
| Modified residue | 369 | Phosphotyrosine | “Quantitative phosphoproteomic analysis of T cell receptor signaling reveals system-wide modulation of protein-protein interactions.” Mayya V., Lundgren D.H., Hwang S.-I., Rezaul K., Wu L., Eng J.K., Rodionov V., Han D.K. Sci. Signal. 2:RA46-RA46(2009) |

The soluble form of ST6Ga11 derives from the membrane form by proteolytic processing.

In some embodiments, one or more of the amino acids of the ST6Ga11 corresponding to amino acids 142, 149, 161, 184, 189, 212, 233, 335, 353, 354, 364, 365, 369, 370, 376, or 406 of ST6Ga11 isoform a (SEQ ID NO: 14) is conserved as compared to SEQ ID NO: 14.

Also provided herein is an enzymatically active portion of, e.g., ST6Ga11. In some embodiments, the enzyme is an enzymatically active portion of STG6Ga11 isoform a (SEQ ID NO: 14), or an ortholog, mutant, or variant of SEQ ID NO: 14. In some embodiments, the enzyme is an enzymatically active portion of STG6Gal1 isoform b (SEQ ID NO: 15), or an ortholog, mutant, or variant of SEQ ID NO: 15.

In some embodiments, the enzymatically active portion of ST6Gal1 does not comprise a cytoplasmic domain, e.g., SEQ ID NO: 16. In some embodiments, the enzymatically active portion of ST6Ga11 does not comprise a transmembrane domain, e.g., SEQ ID NO: 17. In some embodiments, the enzymatically active portion of ST6Ga11 does not comprise a cytoplasmic domain, e.g., SEQ ID NO: 16 or a transmembrane domain, e.g., SEQ ID NO: 17.

In some embodiments, the enzymatically active portion of ST6Gal1 comprises all or a portion of a luminal domain, e.g., SEQ ID NO: 18, or an ortholog, mutants, or variants thereof.

In some embodiments, the enzymatically active portion of ST6Gal1 comprises amino acids 87-406 of SEQ ID NO: 14 (SEQ ID NO: 19), or an ortholog, mutants, or variants thereof. In some embodiments, the enzymatically active portion of ST6Ga11 consists of SEQ ID NO: 19, or an ortholog, mutant, or variant of SEQ ID NO: 19.

A suitable functional portion of an ST6Gal1 can comprise or consist of an amino acid sequence that is at least 80% (85%, 90%, 95%, 98% or 100%) identical to SEQ ID NO: 19.

In some embodiments, the ST6Ga11 comprises or consists of SEQ ID NO: 19, the portion of SEQ ID NO: 19 from amino acid 4 to 320, or the portion of SEQ ID NO: 19 from amino acid 5 to 320.

Also suitable for use in the methods described herein is an amino acid sequence that comprises or consists of an amino acid sequence that is at least 80% (85%, 90%, 95%, 98% or 100%) identical to SEQ ID NO: 20.

### Antibodies

The methods described herein can include galactosylation and sialylation of antibodies. Suitable antibodies include, for example, IgG antibodies. The antibodies, e.g., IgG antibodies, can be pooled. For example, pooled IgG antibodies include IVIg.

In some embodiments, the IgG antibodies comprise IgG antibodies isolated from at least 1000 donors.

In some embodiments, at least 50%, 55%, 60%, 65% or 70% w/w of the IgG antibodies are IgG1 antibodies.

In some embodiments, at least 90% of the donor subject has been exposed to a virus.

In some embodiments, the methods described herein include providing a mixture of IgG antibodies. In some embodiments, providing a mixture of IgG antibodies includes (a) providing pooled plasma from at least 1000 human subjects; and (b) isolating a mixture of IgG antibodies from the pooled plasma. In some embodiments, the mixture of IgG antibodies are isolated from intravenous immunoglobulin. In some embodiments, the mixture of IgG antibodies are intravenous immunoglobulin. In some embodiments, the step of isolating a mixture of IgG antibodies from the pooled plasma comprises ethanol precipitation or caprylic acid (also called octanoic acid) precipitation. In some embodiments, the step of isolating a mixture of IgG antibodies from the pooled plasma comprises biding IgG antibodies to an ion exchange column and eluting the IgG antibodies from an ion exchange column.

### Enzymatic Galactosylation and Sialylation

The methods described herein can comprise a galactosylation step. An exemplary galactosylation reaction is depicted in FIG. 3. Thus, provided herein is a method for galactosylating antibod(ies), e.g., antibod(ies) described herein, by providing a composition (a galactosylation mixture) comprising: antibod(ies), e.g., antibod(ies) described herein; a galactosylating enzyme, e.g., a galactosylating enzyme described herein, e.g., B4GalT or enzymatically active portion of variant thereof; UDP-gal or salt thereof; and incubating the composition under conditions effective for galactosylating the antibody, e.g., as described herein, thereby producing galactosylated antibod(ies).

The methods described herein can comprise a sialylation step. An exemplary sialylation reaction is depicted in FIG. 3. Thus, provided herein is a method for sialylating, e.g., hyper-sialylating, antibod(ies), e.g., antibod(ies) described herein, by providing a composition (a sialylation reaction mixture) comprising: galactosylated antibod(ies), e.g., as described herein; a sialylating enzyme, e.g., a sialylating enzyme described herein, e.g., ST6Gal1 or enzymatically active portion or variant thereof; CMP-NANA or a salt thereof; and incubating the composition under conditions effective for sialylating the antibod(ies), e.g., as described herein.

In some embodiments, the galactosylation step and the sialylation step are carried out sequentially in the same reaction mixture, that is, the galactosylation reaction mixture becomes the sialylation reaction mixture upon addition of the sialylating enzyme and CMP-NANA or salt thereof. In some embodiments, there galactosylation reaction mixture is not filtered, fractionated, or purified prior to the sialylation step. In some embodiments, the galactosylation step and the sialylation step are carried out separately, e.g, pre-galactosylated antibod(ies) are provided, though they may have been processed (e.g., filtered, fractionated, or purified) and/or stored prior to the sialylation step.

Thus, the methods described herein can also comprise a sequential galactosylation and sialylation step. An exemplary galactosylation and sialylation reaction is depicted in FIG. 3. Thus, provided herein is a method for galactosylating and sialylating, e.g., hyper-sialylating, antibod(ies), e.g., antibod(ies) described herein, by a) providing a composition (a galactosylation reaction mixture) comprising: antibod(ies), e.g., as described herein; a galactosylating enzyme, e.g., a galactosylating enzyme described herein, e.g., B4GalT or enzymatically active portion or variant thereof; UDP-gal or a salt thereof; and b) incubating the composition under conditions effective for galactosylating the antibod(ies), e.g., as described herein; c) adding a sialylating enzyme, e.g., a sialylating enzyme described herein, e.g., ST6Gal1 or enzymatically active portion or variant thereof and CMP-NANA or salt thereof to the galactosylation reaction mixture, thereby producing a sialylation reaction mixture; and d) incubating the composition under conditions effective for sialylating the galactosylated antibod(ies), e.g., as described herein.

Also provided herein is a method for galactosylating and sialylating, e.g., hyper-sialylating antibod(ies), e.g., antibod(ies) described herein, by providing a composition comprising: antibod(ies), e.g., as described herein; a galactosylating enzyme, e.g., a galactosylating enzyme described herein, e.g., B4GalT or enzymatically active portion or variant thereof; UDP-gal or a salt thereof; a sialylating enzyme, e.g., a sialylating enzyme described herein, e.g., ST6Gal1 or enzymatically active portion or variant thereof; CMP-NANA or salt thereof; and d) incubating the composition under conditions effective for galactosylating and sialylating the antibod(ies), e.g., as described herein.

In some embodiments, the galactosylation reaction mixture and/or the sialylation reaction mixture comprises Bis (2-hydroxyethyl) aminotris (hydroxymethyl)methane (BIS-TRIS) buffer.

In some embodiments, the galactosylation reaction mixture and/or the sialylation reaction mixture comprises MnCl₂.

In some embodiments, one or more component(s) of one or more of the reaction mixture(s) are supplemented during the incubation. That is, the reaction mixture may comprise an amount of the component at the beginning of the reaction (which may change during the course of the reaction), but also be supplemented with additional amounts of the component(s) during the reaction.

In some embodiments, the B4GalT comprises or consists of an amino acid sequence is at least 90% identical SEQ ID NO: 12 or SEQ ID NO: 13.

In some embodiments, the ST6Ga11 comprises or consists of an amino acid sequence that is at least 90% identical SEQ ID NO: 19 or SEQ ID NO: 20.

In some embodiments, at least or about 60%, 65%, 70%, 75%, 80%, or 85% of the branched glycans on the sialylated antibod(ies), e.g., hsIgG, have a sialic acid on both the α1,3 branch and the α1,6 branch.

In some embodiments, about or at least 60%, 65%, 70%, 75%, 80%, or 85% of the branched Fc glycans on the sialylated antibod(ies), e.g., hsIgG, have a sialic acid on both the α1,3 branch and the α1,6 branch.

In some embodiments, about or at least 60%, 65%, 70%, 75%, 80%, or 85% of the branched glycans on the Fab domain of the sialylated antibod(ies), e.g., hsIgG, have a sialic acid on both the α 1,3 arm and the α 1,6 arm that is connected through a NeuAc-α 2,6-Gal terminal linkage.

In some embodiments, about or at least 80% of the branched Fc glycans on the sialylated antibod(ies), e.g., hsIgG, have a sialic acid on both the α1,3 branch and the α1,6 branch.

In some embodiments, about or at least 60%, 65%, 70% of the branched glycans on the Fab domain of the sialylated antibod(ies), e.g., hsIgG, have a sialic acid on both the α 1,3 arm and the α 1,6 arm that is connected through a NeuAc-α 2,6-Gal terminal linkage.

In some embodiments, about or at least 85% of the of the branched Fc glycans on the sialylated antibod(ies), e.g., hsIgG, have a sialic acid on both the α1,3 branch and the α1,6 branch.

In some embodiments, about or at least 60%, 65%, 70% of the branched glycans on the Fab domain of the sialylated antibod(ies), e.g., hsIgG, have a sialic acid on both the α 1,3 arm and the α 1,6 arm that is connected through a NeuAc-α 2,6-Gal terminal linkage.

In some embodiments, about or at least 90% of the of the branched Fc glycans on the sialylated antibod(ies), e.g., hsIgG, have a sialic acid on both the α1,3 branch and the α1,6 branch.

In some embodiments, about or at least 60%, 65%, 70% of the branched glycans on the Fab domain of the sialylated antibod(ies), e.g., hsIgG, have a sialic acid on both the α 1,3 arm and the α 1,6 arm that is connected through a NeuAc-α 2,6-Gal terminal linkage.

### Isolation and Purification Methods

Provided herein are methods for purifying antibod(ies), e.g., IgG, e.g., pooled IgG, e.g., IVIg, e.g., sialylated IgG, e.g., sialylated pooled IgG, e.g., sialylated IVIg, e.g., hsIgG

Also provided herein are methods for producing purified sialylated antibod(ies), e.g., purified hsIgG, the method comprising: i) producing sialylated antibod(ies), e.g., hsIgG, e.g., as described herein; and ii) purifying sialylated antibod(ies), e.g., as described herein.

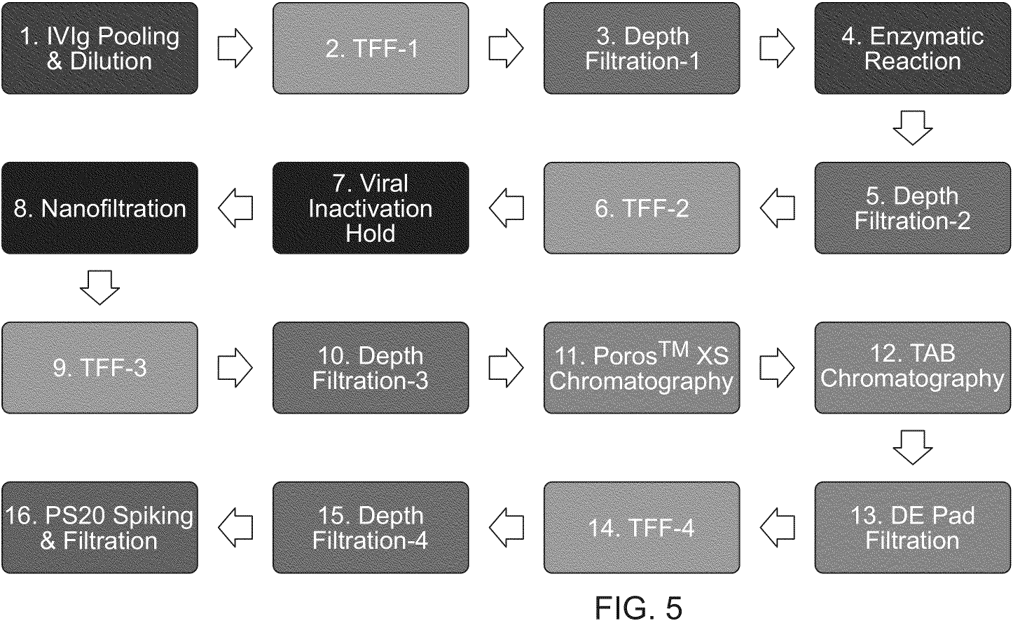

An exemplary manufacturing process for isolation and purification of hsIgG is shown in FIG. 5. Variations of that process are described herein.

In some embodiments, the methods for purifying antibod(ies) comprises analyzing the amount of one or more IgG subclasses after purification, e.g., after a chromatography step.

### Quenching

The methods described herein can include quenching of an enzymatic reaction, e.g., an enzymatic sialylation reaction, e.g., as described herein.

In some embodiments, quenching comprises mixing the composition comprising sialylated antibod(ies), e.g., sialylation reaction mixture, e.g., as described herein, with a buffer, e.g., phosphate buffered saline (PBS), e.g., 5X PBS, thereby producing a quenched antibody composition. In some embodiments, quenching comprises mixing the composition and the buffer at or at about 1:1. In some embodiments, quenching comprises mixing the antibody composition and the buffer at 1 volume buffer or more to one volume antibody composition.

### Depth Filtration

The methods described herein can include depth filtration. Depth filtration is well known in the art and described, e.g., in Sutherland, “Filtration Overview: A Closer Look at Depth Filtration,” Filtration & Separation 45(8):25-28 (2008); Nguyen et al., “Improved HCP Reduction Using a New, All-Synthetic Depth Filtration Media Within an Antibody Purification Process,” Biotechnology Journal 14(1):1700771 (2019).

In some embodiments, the methods described herein, e.g., methods for producing and/or purifying antibod(ies), e.g., IgG, e.g., pooled IgG, e.g., IVIg, e.g., e.g., sialylated IgG, e.g., sialylated pooled IgG, e.g., sialylated IVIg, e.g., hsIgG, include one or more depth filtration steps. In some embodiments, the method includes one or more depth filtration steps before a chromatography step. In some embodiments, the method includes one or more depth filtration steps after a chromatography step. In some embodiments, the method does not include a depth filtration step prior to chromatography. In some embodiments, the method does not include a depth filtration step after chromatography. In some embodiments, the method does not include a depth filtration step.

### Buffer Exchange

The methods described herein can include buffer exchange. Methods for buffer exchange are well known in the art and described, e.g., in Kurnik, “Buffer Exchange Using Size Exclusion Chromatography, Countercurrent Dialysis, and Tangential Flow Filtration: Models, Development, and Industrial Application,” Biotechnology & Bioengineering 45(2): 149-58 (1995); Dizon-Maspat et al., “Single Pass Tangential Flow Filtration to Debottleneck Downstream Processing for Therapeutic Antibody Production,” Biotechnology & Bioengineering 109(4):962-970 (2012).

In some embodiments, the buffer exchange method is size exclusion chromatography (SEC). In some embodiments, the buffer exchange method is tangential flow filtration (TFF). In some embodiments, the buffer exchange method is countercurrent dialysis (CCD).

In some embodiments, the buffer exchange method is tangential flow filtration (TFF).

In some embodiments, the methods described herein, e.g., methods for producing and/or purifying antibod(ies), e.g., IgG, e.g., pooled IgG, e.g., IVIg, e.g., e.g., sialylated IgG, e.g., sialylated pooled IgG, e.g., sialylated IVIg, e.g., hsIgG, include one or more buffer exchange steps. In some embodiments, the method includes one or more buffer exchange steps before a chromatography step. In some embodiments, the method includes one or more buffer exchange steps after a chromatography step. In some embodiments, the method does not include a buffer exchange step prior to chromatography. In some embodiments, the method does not include a buffer exchange step after chromatography. In some embodiments, the method does not include a buffer exchange step.

### Viral Inactivation/Removal

The methods described herein can include viral inactivation or viral removal. Methods for viral inactivation and removal are well known in the art and described, e.g., in Horowitz et al., “Strategies for Viral Inactivation,” Current Opinion in Hematology 2(6):484-92 (1995); Klutz et al., “Continuous Viral Inactivation at low pH Value in Antibody Manufacturing,” Chemical Engineering and Processing: Process Intensification 102:88-11 (2016).

In some embodiments, the viral inactivation method is selected from solvent/detergent inactivation, low pH inactivation, pasteurization, microwave heating, irradiation, high-energy light, and combinations thereof.

In some embodiments, the viral removal method is selected from affinity chromatography, nanofiltration, and combinations thereof.

In some embodiments, the methods described herein, e.g., methods for purification of antibod(ies), e.g., IgG, e.g., pooled IgG, e.g., IVIg, e.g., e.g., sialylated IgG, e.g., sialylated pooled IgG, e.g., sialylated IVIg, e.g., hsIgG, include one or more viral inactivation or viral removal steps. In some embodiments, the viral inactivation or viral removal step is carried out after galactosylation and sialylation. In some embodiments, the viral inactivation or viral removal step is carried out after galactosylation and sialylation but before chromatography. In some embodiments, the viral inactivation and removal includes viral inactivation followed by nanofiltration. In some embodiments, the method comprises viral removal (e.g., by nanofiltration), but does not include a separate viral inactivation step.

### Nanofiltration

The methods described herein can include nanofiltration. Membranes and methods for nanofiltration are well known in the art and described, e.g., in Mohammad et al., “Nanofiltration Membranes Review: Recent Advances and Future Prospects,” Desalination 356:226-54 (2015).

In some embodiments, the methods described herein, e.g., methods for producing and/or purifying antibod(ies), e.g., IgG, e.g., pooled IgG, e.g., IVIg, e.g., e.g., sialylated IgG, e.g., sialylated pooled IgG, e.g., sialylated IVIg, e.g., hsIgG, include one or more nanofiltration steps. In some embodiments, the method includes one or more nanofiltration steps before a chromatography step. In some embodiments, the method includes one or more nanofiltration steps after a chromatography step. In some embodiments, the method does not include a nanofiltration step prior to chromatography. In some embodiments, the method does not include a nanofiltration step after chromatography. In some embodiments, the method does not include a nanofiltration step.

### Chromatography

The methods described herein e.g., methods for producing and/or purifying antibod(ies), e.g., IgG, e.g., pooled IgG, e.g., IVIg, e.g., e.g., sialylated IgG, e.g., sialylated pooled IgG, e.g., sialylated IVIg, e.g., hsIgG, can include chromatography as part of the purification process.

In some embodiments, the antibod(ies) bind to the chromatography resin. In some embodiments, the antibod(ies) flow through the chromatography resin.

In some embodiments, the impurity(ies) (e.g., one or more of the other components of the enzymatic reaction mixture(s), e.g., galactosylation reaction mixture(s) and/or sialylation reaction mixture(s), including, but not limited to, enzymes, e.g., B4GalT and/or ST6, nucleotide sugars, and MnCl₂) bind to the chromatography resin. In some embodiments, the impurities flow through the chromatography resin.

In some embodiments, both the antibod(ies) and the impurity(ies) bind to the chromatography resin. In such an embodiment, the antibod(ies) and impurity(ies) can be separately eluted.

### Protein A Affinity Chromatography

The methods described herein e.g., methods for producing and/or purifying antibod(ies), e.g., IgG, e.g., pooled IgG, e.g., IVIg, e.g., e.g., sialylated IgG, e.g., sialylated pooled IgG, e.g., sialylated IVIg, e.g., hsIgG, can include an affinity chromatography step in which the antibod(ies) bind to the ligand of the chromatography resin, e.g., a Protein A affinity chromatography step.

Materials and methods for Protein A affinity chromatography are well known in the art and described, e.g., in Ramos-de-la-Peña et al., “Protein A chromatography: Challenges and Progress in the Purification of Monoclonal Antibodies,” Journal of Separation Science 2019:doi: 10.1002/jssc.201800963.

In some embodiments, the Protein A affinity chromatography resin is MabSelect™ PrismA (GE Healthcare).

In some embodiments, the Protein A affinity chromatography resin ligand is selected from the group consisting of Protein A, a Protein A derivative, a Protein A mimetic, or a combination thereof.

In some embodiments, the methods describe herein comprise Protein A Affinity Chromatography followed by Blue Dye Chromatography, e.g., as described herein.

### Cation Exchange Chromatography

The methods described herein e.g., methods for producing and/or purifying antibod(ies), e.g., IgG, e.g., pooled IgG, e.g., IVIg, e.g., e.g., sialylated IgG, e.g., sialylated pooled IgG, e.g., sialylated IVIg, e.g., hsIgG, can include a cation exchange chromatography (CEX) step, e.g., a strong cation exchange chromatography step using a strong cation exchange chromatography resin.

In some embodiments, the strong cation exchange resin is selected from the group consisting of Poros™ XS (e.g., Cytiva 4404336), SP Sepharose High Performance (SP HP) (e.g., Cytiva 28950515), Capto™ SP Impres (e.g., Cytiva 17546815), Capto™ S (e.g., Cytiva 28926979), Capto™ S Impact (e.g., Cytiva 1737174), and combinations thereof.

The Poros™ strong cation exchange (CEX) resins are 50-µm, rigid, polymeric, ionexchange chromatography resins. Thermoscientific Product Information Sheet Pub. No. 100031321 Rev. D. A polyhydroxyl surface coating provides low non-specific binding and surface functionalization with sulphopropyl yields a strong cation-exchanger ionizable with pH 1-14. Id. The support matrix is cross-linked polystyrene-divinylbenzene and the surface functionality is sulfopropyl (—CH₂CH₂CH₂SO₃^{—}). Id. The dynamic binding capacity of Poros™ XS is ≥ 102 mg/mL (5% breakthrough of Polyclonal Human IgG in 20 mM MES< 40 mM NaCl, pH 5.0 at 300 cm/hour in 0.46 cmD × 20 cmL column). Id. The dynamic binding capacity of Poros™ 50 HS 57.0-75.3 mg/mL (5% breakthrough with Lysozyme in 20 mM MES, pH 6.2 at 100 cm/hour in 0.46 cmD × 20 cmL column). Id.

The SP Sepharose High Performance (SP HP) resin is based on a 6% cross-linked agarose matrix with a particle size of 24-44 µm (d₅₀ᵥ, ~34 µm) that uses a sulphopropyl ligand (—CH₂CH₂CH₂SO₃^{—}). See Product Bulleting “SP Sepharose High Performance”, cytiva.com. It has an operational pH stability of 4-13. Id. It has a dynamic binding capacity of ~ 55 ribonuclease A/mL resin. Id.

Capto™ SP ImpRes is based on a high-flow agarose base matrix with a bead size of about 40 µm. Product Bulletin “Capto™ SP ImpRes, Capto™ Q ImpRes Ion Exchange Chromatography”, cytiva.com. The functional group of Capto™ SP Impres is —CH₂CH₂CH₂SO₃^{—}. Id. The working operating pH stability is 4 to 12. The binding capacity (10% breakthrough measured at a residence time of 4 min (150 cm/h) in a TricornTM 5/100 column with 10 cm bed height; 20 mM sodium phosphate, pH 7.2 (lysozyme) and 50 mM Tris, pH 8.0 (BSA)) is > 70 mg lysozyme/mL medium and > 95 mg BSA/mL medium.

Capto™ S is a strong cation exchange resin based on a highly cross-linked agarose, spherical matrix with a median particle size of the cumulative volume distribution of about 90 µm that uses the —SO3^{—} charged group shown below

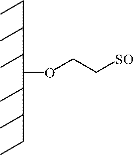

See Cytiva, “Ion Exchange Resins”, 28407452 AG, cytiva.com. It has a dynamic binding capacity of > 120 mg lysozyme / mL resin (at 10% breakthrough by frontal analysis at a mobile phase velocity of 600 cm/h in a Tricorn™ 5/100 column at 10 cm bed height (1 min residence time) for lysozyme in 30 mM sodium phosphate, pH 6.8) and > 60 mg β-Lactoglobulin/mL resin (at 10% breakthrough by frontal analysis at a mobile phase velocity of 600 cm/h in a PEEK 7.5/100 column at 10 cm bed height (1 min residence time) for β-lactoglobulin in 100 mM citrate, pH 3) and can be operated at pH 4 to 12.

The Capto™ S Impact is based on a high-flow agarose base matrix with a median particle size of the cumulative volume distribution of about 50 µm that uses a —SO3^{—} ligand. See Capto™ S ImpAct product sheet, cytiva.com. It has a binding capacity of >85 mg BSA/mL resin, >90 mg lysozyme/mL resin, >100 mg IgG/ mL resin. Id.

In some embodiments, the strong cation exchange resin has a binding capacity of 50 mg/mL or more, e.g., 75 mg/mL or more, e.g., 100 mg/mL or more, measured as 5% breakthrough of Polyclonal Human IgG in 20 mM MES< 40 mM NaCl, pH 5.0 at 300 cm/hour in 0.46 cmD × 20 cmL column.

In some embodiments, the strong cation exchange resin maintains binding capacity of 50 mg/mL or more, e.g., 75 mg/mL or more, e.g., 100 mg/mL or more, measured as 5% breakthrough of Polyclonal Human IgG in 20 mM MES< 40 mM NaCl, pH 5.0 at 300 cm/hour in 0.46 cmD × 20 cmL column, at up to 150 mM NaCl (15 mS/cm).

In some embodiments, the functional group of the strong cation exchange resin comprises SO₃—. In some embodiments, the functional group of the strong cation exchange resin is —CH₂CH₂CH₂SO₃^{—}.

In some embodiments, the cation exchange chromatography is carried out in flow through mode. In some embodiments, the cation exchange chromatography is carried out in bind and elute mode. In some embodiments, the antibod(ies), e.g., sialylated antibod(ies), e.g., hsIgG, flow through the cation exchange chromatography resin. In some embodiments, the antibod(ies), e.g., sialylated antibod(ies), e.g., hsIgG, are bound and then eluted from the cation exchange chromatography resin.

In some embodiments, a composition comprising sialylated antibod(ies), e.g., hsIgG, and one or more impurities is loaded on to the CEX column. In some embodiments the composition is a sialylation reaction mixture, e.g., a sialylation mixture comprising sialylated antibod(ies), e.g., as described herein, is loaded on to the CEX column.

In some embodiments, from or from about 10 to or to about 110 mg antibod(ies), per mL resin are loaded on to the CEX column. In some embodiments, from or from about 10 to or to about 100, from or from about 10 to or to about 90, from or from about 10 to or to about 80, from or from about 10 to or to about 70, from or from about 10 to or to about 60, from or from about 10 to or to about 50, from or from about 10 to or to about 40, from or from about 10 to or to about 30, from or from about 10 to or to about 20, from or from about 20 to or to about 110, from or from about 20 to or to about 100, from or from about 20 to or to about 90, from or from about 20 to or to about 80, from or from about 20 to or to about 70, from or from about 20 to or to about 60, from or from about 20 to or to about 50, from or from about 20 to or to about 40, from or from about 20 to or to about 30, from or from about 30 to or to about 110, from or from about 30 to or to about 100, from or from about 30 to or to about 90, from or from about 30 to or to about 80, from or from about 30 to or to about 70, from or from about 30 to or to about 60, from or from about 30 to or to about 50, from or from about 30 to or to about 40, from or from about 40 to or to about 110, from or from about 40 to or to about 100, from or from about 40 to or to about 90, from or from about 40 to or to about 80, from or from about 40 to or to about 70, from or from about 40 to or to about 60, from or from about 40 to or to about 50, from or from about 50 to or to about 110, from or from about 50 to or to about 100, from or from about 50 to or to about 90, from or from about 50 to or to about 80, from or from about 50 to or to about 70, from or from about 50 to or to about 60, from or from about 60 to or to about 110, from or from about 60 to or to about 100, from or from about 60 to or to about 90, from or from about 60 to or to about 80, from or from about 60 to or to about 70, from or from about 70 to or to about 110, from or from about 70 to or to about 100, from or from about 70 to or to about 90, from or from about 70 to or to about 80, from or from about 80 to or to about 110, from or from about 80 to or to about 100, from or from about 80 to or to about 90, from or from about 90 to or to about 110, from or from about 90 to or to about 100, or from or from about 100 to or to about 110 mg antibod(ies), e.g., sialylated antibod(ies), e.g., hsIgG per mL resin are loaded on to the CEX column.

In some embodiments, the methods described herein include one or more of: (a) a quenching step, e.g., as described herein, (b) a depth filtration step, e.g., as described herein (c) a buffer exchange step, e.g., as described herein, (d) a virus inactivation or removal step, e.g., as described herein, and (e) a nanofiltration step, e.g., as described herein, before CEX chromatography.

In some embodiments, the methods described herein comprise one or more of: (a) blue dye chromatography, e.g., as described herein, (b) DE pad filtration, e.g., as described herein, (c) buffer exchange, e.g., as described herein, and (d) PS20 Spiking & Filtration, e.g., as described herein, after CEX chromatography.

Provided herein is a method for purifying sialylated antibod(ies), which includes, in order from (a) to (d): (a) providing a composition comprising sialylated antibod(ies), e.g., hsIgG and galactosylation and/or sialylation enzyme(s), e.g., B4GalT and/or ST6 or enzymatically active portion thereof, e.g., as described herein, and, for example, if it is a sialylation reaction mixture, e.g., as described herein, optionally quenching the reaction, e.g., as described herein, e.g., in PBS, e.g., in 5X PBS at a 1:1 dilution; (b) diluting the composition, e.g., with citrate buffer (e.g., 6.757 g/L sodium citrate dehydrate and 5.192 g/L citric acid buffer), e.g., citrate buffer at or at about 50 mM, at or at about pH 4.5, thereby producing a buffered antibody composition; (c) applying the buffered antibody composition to a CEX column, e.g., a CEX column described herein, under conditions that bind the sialylated antibod(ies), e.g., hsIgG, as well as the galactosylation and/or sialylation enzyme(s); and (d) selectively eluting the sialylated antibod(ies), e.g., hsIgG from the CEX column, thereby producing purified sialylated antibod(ies).

In some embodiments, the method does not contain any additional filtering, fractionation, or purification steps other than (a) and (b) before carrying out step (c). In some embodiments, the method comprises a single depth filtering step, in addition to steps (a) and (b) before step (c), e.g., between steps (a) and (b), but does not comprise any additional filtering, fractionation, or purification steps beyond the single depth filtration step before carrying out step (c). In some embodiments, the method comprises a viral inactivation step before step (c). In some embodiments, the applying in step (c) is spaced out in time, e.g., a portion of the buffered antibody composition is applied to the column over more than one application, e.g., over 1, 2, or 3 applications. In some embodiments, step (e) is repeated one or more times. In some embodiments, step (c) is carried out at resident time(s) of from or from about 1, 2, 3, 4, 5, or 6 minutes.

In some embodiments, selectively eluting the sialylated antibod(ies) comprises eluting in a buffer comprising 400 mM or more NaCl, e.g., a citrate buffer at or at about 50 mM, 400 mM or more NaCl, at or at about pH 4.5.

In some embodiments, the method further comprises, following step (e), one or more of: (f) a blue dye chromatography step, e.g., a Trisacyl Blue (TAB) chromatography, e.g., as described herein, (g) a DE Pad filtration step, e.g., as described herein, (h) a depth filtration step, e.g., as described herein; and (g) a PS20 spiking & filtration step, e.g., as described herein.

In some embodiments, this method produces yields (antibody recovery rate) of or of about 80%, 85%, 90%, or 95% or more with sialylating enzyme, e.g., ST6 or enzymatically active portion thereof, present at 100 ppm, 90 ppm, 80 ppm, 70 ppm, 60 ppm, 50 ppm, 40 ppm, or 30 ppm or less.

### Blue Dye Affinity Chromatography

The methods described herein e.g., methods for producing and/or purifying antibod(ies), e.g., IgG, e.g., pooled IgG, e.g., IVIg, e.g., e.g., sialylated IgG, e.g., sialylated pooled IgG, e.g., sialylated IVIg, e.g., hsIgG, can include blue dye affinity chromatography as part of the purification process, e.g., an affinity chromatography step using a blue dye resin.

In some embodiments, the blue dye resin is selected from the group consisting of Blue Trisacryl™ (e.g., Sartorius 25896-010), Capto™ Blue (e.g., Cytiva 17544801), Capto™ Blue HS, (e.g., Cytiva 17545202), Blue Sepharose™ (e.g., Cytiva 17-0948-01), and combinations thereof.

The Blue Trisacryl™ resin is based on a synthetic polymer matrix with a bead size of 60 µm that uses a blue dye ligand as the functional group. See Product Specifications, Sartorius 25896-010, Sartorius.com. It has an operational pH of 4-10. Id.

The Capto™ Blue resins are based on a highly cross-linked agarose matrix with an average particle size of 75 µm. See Product Bulletin, “Capto Blue and Capto Blue (high sub) Affinity Chromatography,” cytiva.com. They use a Cibacron™ Blue (1-amino-4-[4-[[4-chloro-6-(2-sulfoanilino)-1,3,5-triazin-2-yl]amino]-3-sulfoanilino]-9,10-dioxoanthracene-2-sulfonic acid) ligand. Id; PubChem ID 172469. They have an operation pH of 3-13. Capto™ Blue has a dynamic binding capacity of HSA at 10% breakthrough of 24 mg/mL at 4 min residence time and Capto™ Blue HS has a dynamic binding capacity of HSA at 10% breakthrough of 30 mg/mL at 4 min residence time. Id.

Blue Sepharose™ is based on a highly cross-linked agarose base matrix (6% spherical) with a median particle size of the cumulative volume distribution of ~90 µm. See Product Bulletin, “Blue Sepharose 6 Fast Flow Affinity Chromatography,” cytiva.com. It uses a Cibacron™ Blue 3G (1-amino-4-[4-[[4-chloro-6-(3-sulfoanilino)-1,3,5-triazin-2-yl]amino]-3-sulfoanilino]-9,10-dioxoanthracene-2-sulfonic acid). It has an operational pH stability from 4-12 and a total binding capacity of ≥ 18 mg human serum albumin/mL resin (Protein in excess is loaded in 0.050 M Potassium dihydrogen phosphate, pH 7.0 on a PEEK 7.5/50 column. The binding capacity is obtained by measuring the amount of bound and eluted protein in 0.050 M Potassium dihydrogen phosphate, 1.5 M Potassium chloride, pH 7.0.).

In some embodiments, the blue dye affinity chromatography is carried out in flow through mode. In some embodiments, the antibod(ies), e.g., sialylated antibod(ies), e.g., hsIgG, flow through the blue dye chromatography resin.

In some embodiments, a composition comprising sialylated antibod(ies), e.g., hsIgG, and one or more impurities is loaded on to the blue dye column. In some embodiments the composition is a sialylation reaction mixture, e.g., a sialylation mixture comprising sialylated antibod(ies), e.g., as described herein, is loaded on to the blue dye column.

In some embodiments, from or from about 10 to or to about 110 mg antibod(ies), per mL resin are loaded on to the blue dye column. In some embodiments, from or from about 10 to or to about 100, from or from about 10 to or to about 90, from or from about 10 to or to about 80, from or from about 10 to or to about 70, from or from about 10 to or to about 60, from or from about 10 to or to about 50, from or from about 10 to or to about 40, from or from about 10 to or to about 30, from or from about 10 to or to about 20, from or from about 20 to or to about 110, from or from about 20 to or to about 100, from or from about 20 to or to about 90, from or from about 20 to or to about 80, from or from about 20 to or to about 70, from or from about 20 to or to about 60, from or from about 20 to or to about 50, from or from about 20 to or to about 40, from or from about 20 to or to about 30, from or from about 30 to or to about 110, from or from about 30 to or to about 100, from or from about 30 to or to about 90, from or from about 30 to or to about 80, from or from about 30 to or to about 70, from or from about 30 to or to about 60, from or from about 30 to or to about 50, from or from about 30 to or to about 40, from or from about 40 to or to about 110, from or from about 40 to or to about 100, from or from about 40 to or to about 90, from or from about 40 to or to about 80, from or from about 40 to or to about 70, from or from about 40 to or to about 60, from or from about 40 to or to about 50, from or from about 50 to or to about 110, from or from about 50 to or to about 100, from or from about 50 to or to about 90, from or from about 50 to or to about 80, from or from about 50 to or to about 70, from or from about 50 to or to about 60, from or from about 60 to or to about 110, from or from about 60 to or to about 100, from or from about 60 to or to about 90, from or from about 60 to or to about 80, from or from about 60 to or to about 70, from or from about 70 to or to about 110, from or from about 70 to or to about 100, from or from about 70 to or to about 90, from or from about 70 to or to about 80, from or from about 80 to or to about 110, from or from about 80 to or to about 100, from or from about 80 to or to about 90, from or from about 90 to or to about 110, from or from about 90 to or to about 100, or from or from about 100 to or to about 110 mg antibod(ies), e.g., sialylated antibod(ies), e.g., hsIgG per mL resin are loaded on to the blue dye column.

In some embodiments, from or from about 10 to or to about 110 centigrams antibod(ies), per mL resin are loaded on to the blue dye column. In some embodiments, from or from about 10 to or to about 100, from or from about 10 to or to about 90, from or from about 10 to or to about 80, from or from about 10 to or to about 70, from or from about 10 to or to about 60, from or from about 10 to or to about 50, from or from about 10 to or to about 40, from or from about 10 to or to about 30, from or from about 10 to or to about 20, from or from about 20 to or to about 110, from or from about 20 to or to about 100, from or from about 20 to or to about 90, from or from about 20 to or to about 80, from or from about 20 to or to about 70, from or from about 20 to or to about 60, from or from about 20 to or to about 50, from or from about 20 to or to about 40, from or from about 20 to or to about 30, from or from about 30 to or to about 110, from or from about 30 to or to about 100, from or from about 30 to or to about 90, from or from about 30 to or to about 80, from or from about 30 to or to about 70, from or from about 30 to or to about 60, from or from about 30 to or to about 50, from or from about 30 to or to about 40, from or from about 40 to or to about 110, from or from about 40 to or to about 100, from or from about 40 to or to about 90, from or from about 40 to or to about 80, from or from about 40 to or to about 70, from or from about 40 to or to about 60, from or from about 40 to or to about 50, from or from about 50 to or to about 110, from or from about 50 to or to about 100, from or from about 50 to or to about 90, from or from about 50 to or to about 80, from or from about 50 to or to about 70, from or from about 50 to or to about 60, from or from about 60 to or to about 110, from or from about 60 to or to about 100, from or from about 60 to or to about 90, from or from about 60 to or to about 80, from or from about 60 to or to about 70, from or from about 70 to or to about 110, from or from about 70 to or to about 100, from or from about 70 to or to about 90, from or from about 70 to or to about 80, from or from about 80 to or to about 110, from or from about 80 to or to about 100, from or from about 80 to or to about 90, from or from about 90 to or to about 110, from or from about 90 to or to about 100, or from or from about 100 to or to about 110 centigrams antibod(ies), e.g., sialylated antibod(ies), e.g., hsIgG per mL resin are loaded on to the blue dye column.

In some embodiments, the methods described herein include one or more of: (a) a quenching step, e.g., as described herein, (b) a depth filtration step, e.g., as described herein (c) a buffer exchange step, e.g., as described herein, (d) a virus inactivation or removal step, e.g., as described herein, and (e) a nanofiltration step, e.g., as described herein, or (f) a CEX chromatography step, e.g., as described herein, before blue dye chromatography.

In some embodiments, the method does not comprise a CEX chromatography step.

In some embodiments, the methods described herein comprise one or more of: (a) DE pad filtration, e.g., as described herein, (b) buffer exchange, e.g., as described herein, and (c) PS20 spiking & filtration, e.g., as described herein, after blue dye chromatography.

Provided herein is a method for purifying sialylated antibod(ies), which includes, in order from (a) to (c): (a) providing a composition comprising sialylated antibod(ies), e.g., hsIgG and galactosylation and/or sialylation enzyme(s), e.g., B4GalT and/or ST6 or enzymatically active portion thereof, e.g., as described herein, and, for example, if it is a sialylation reaction mixture, e.g., as described herein, optionally quenching the reaction, e.g., as described herein, e.g., in PBS, e.g., in 5X PBS at a 1:1 dilution; (b) diluting the composition in a buffer suitable for use with the column, e.g., for a TAB column with citrate buffer, e.g., citrate buffer at or at about 100 mM, at or at about pH 4.5 with or with about 800 mM NaCl or for a CaptBlue column in or in about 250 mM glycine plus or plus about 100 mM sodium chloride at or at about pH 4.5, thereby producing a buffered antibody composition; and (c) applying the buffered antibody composition to a blue dye column, e.g., a blue column described herein, under conditions that allow 80% or more, e.g., 85% or more, 90% or more, or 95% or more of the sialylated antibod(ies), e.g., hsIgG to flow through, but allows only 30, 40, 50, 60, 70, 80, 90, or 100 ppm or less of the galactosylation and/or sialylation enzyme(s) to flow through, thereby producing purified sialylated antibod(ies).

In some embodiments, the method does not contain any additional filtering, fractionation, or purification steps other than (a) and (b) before carrying out step (c). In some embodiments, the method comprises a single depth filtering step, in addition to steps (a) and (b) before step (c), e.g., between steps (a) and (b), but does not comprise any additional filtering, fractionation, or purification steps beyond the single depth filtration step before carrying out step (c). In some embodiments, the method comprises a virus inactivation step before step (c). In some embodiments, the applying in step (c) is spaced out in time, e.g., a portion of the buffered antibody composition is applied to the column over more than one application, e.g., over 1, 2, or 3 applications. In some embodiments, step (c) is carried out at resident time(s) of from or from about 1, 2, 3, 4, 5, or 6 minutes.

In some embodiments, the method further comprises, following step (c), one or more of: (d) a CEX chromatography step, e.g., as described herein, (e) a DE pad filtration step, e.g., as described herein, (f) a depth filtration step, e.g., as described herein; and (g) a PS20 spiking & filtration step, e.g., as described herein.

In some embodiments, the method further comprises, following step (c), one or more of: (d) a DE pad filtration step, e.g., as described herein, (e) a depth filtration step, e.g., as described herein; and (f) a PS20 spiking & filtration step, e.g., as described herein, but does not comprise a CEX chromatography step, e.g., as described herein.

In some embodiments, this method produces yields (antibody recovery rate) of or of about 80%, 85%, 90%, or 95% or more with sialylating enzyme, e.g., ST6 or enzymatically active portion thereof, present at 100 ppm, 90 ppm, 80 ppm, 70 ppm, 60 ppm, 50 ppm, 40 ppm, or 30 ppm or less.

### Polysorbate Spiking

Polysorbate, e.g., polysorbate 20, can be added to composition comprising the antibod(ies) described herein, e.g., hsIgG, e.g., purified hsIgG. In some embodiments, the polysorbate is polysorbate 20. In some embodiments, the polysorbate 20 is Super Refined™ polysorbate 20 (Croda Health Care).

### Precipitation

The methods described herein can include precipitation. In some embodiments, the precipitation is ammonium sulfate precipitation.

In some embodiments, the methods described herein, e.g., methods for purification of antibod(ies), e.g., IgG, e.g., pooled IgG, e.g., IVIg, e.g., e.g., sialylated IgG, e.g., sialylated pooled IgG, e.g., sialylated IVIg, e.g., hsIgG, include ammonium sulfate precipitation.

### Variants

In some embodiments, the enzyme(s) described herein are at least 80%, e.g., at least 85%, 90%, 95%, 98%, or 100% identical to the amino acid sequence of an exemplary sequence (e.g., as provided herein), e.g., have differences at up to 1%, 2%, 5%, 10%, 15%, or 20% of the residues of the exemplary sequence replaced, e.g., with conservative mutations, e.g., including or in addition to the mutations described herein. In preferred embodiments, the variant retains desired activity of the parent, e.g., β-galactoside α-2,6-sialyltransferase activity or β-1,4-galactosyltransferase activity.

To determine the percent identity of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The length of a reference sequence aligned for comparison purposes is at least 80% of the length of the reference sequence, and in some embodiments is at least 90% or 100%. The nucleotides at corresponding amino acid positions or nucleotide positions are then compared. When a position in the first sequence is occupied by the same nucleotide as the corresponding position in the second sequence, then the molecules are identical at that position (as used herein nucleic acid “identity” is equivalent to nucleic acid “homology”). The percent identity between the two sequences is a function of the number of identical positions shared by the sequences, taking into account the number of gaps, and the length of each gap, which need to be introduced for optimal alignment of the two sequences.

Percent identity between a subject polypeptide or nucleic acid sequence (i.e. a query) and a second polypeptide or nucleic acid sequence (i.e. target) is determined in various ways that are within the skill in the art, for instance, using publicly available computer software such as Smith Waterman Alignment (Smith, T. F. and M. S. Waterman (1981) J Mol Biol 147:195-7); “BestFit” (Smith and Waterman, Advances in Applied Mathematics, 482-489 (1981)) as incorporated into GeneMatcher Plus™, Schwarz and Dayhof (1979) Atlas of Protein Sequence and Structure, Dayhof, M.O., Ed, pp 353-358; BLAST program (Basic Local Alignment Search Tool; (Altschul, S. F., W. Gish, et al. (1990) J Mol Biol 215: 403-10), BLAST-2, BLAST-P, BLAST-N, BLAST-X, WU-BLAST-2, ALIGN, ALIGN-2, CLUSTAL, or Megalign (DNASTAR) software. In addition, those skilled in the art can determine appropriate parameters for measuring alignment, including any algorithms needed to achieve maximal alignment over the length of the sequences being compared. In general, for target proteins or nucleic acids, the length of comparison can be any length, up to and including full length of the target (e.g., 5%, 10%, 20%, 30%, 40%, 50%, 60%, 70%, 80%, 90%, 95%, or 100%). For the purposes of the present disclosure, percent identity is relative to the full length of the query sequence.

For purposes of the present disclosure, the comparison of sequences and determination of percent identity between two sequences can be accomplished using a Blossum 62 scoring matrix with a gap penalty of 12, a gap extend penalty of 4, and a frameshift gap penalty of 5.

Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine.

### EXAMPLES

The invention is further described in the following examples, which do not limit the scope of the invention described in the claims.

### Example 1: Hypersialylated IgG Preparation

IgG in which more than 60% of the overall branched glycans are disialylated can be prepared as follows.

Briefly, a mixture of IgG antibodies is exposed to a sequential enzymatic reaction using β1,4 galactosyltransferase 1 (B4GalT) and α2,6-sialyltransferase (ST6Gal1) enzymes. The B4GalT does not need to be removed from the reaction before addition of ST6Ga11 and no partial or complete purification of the product is needed between the enzymatic reactions.

The galactosyltransferase enzyme selectively adds galactose residues to pre-existing asparagine-linked glycans. The resulting galactosylated glycans serve as substrates to the sialic acid transferase enzyme which selectively adds sialic acid residues to cap the asparagine-linked glycan structures attached to. Thus, the overall sialylation reaction employed two sugar nucleotides (uridine 5′-diphosphogalactose (UDP-Gal) and cytidine-5′-monophospho-N-acetylneuraminic acid (CMP-NANA)). The latter is replenished periodically to increase disialylated product relative to monosialylated product. The reaction includes the co-factor manganese chloride.

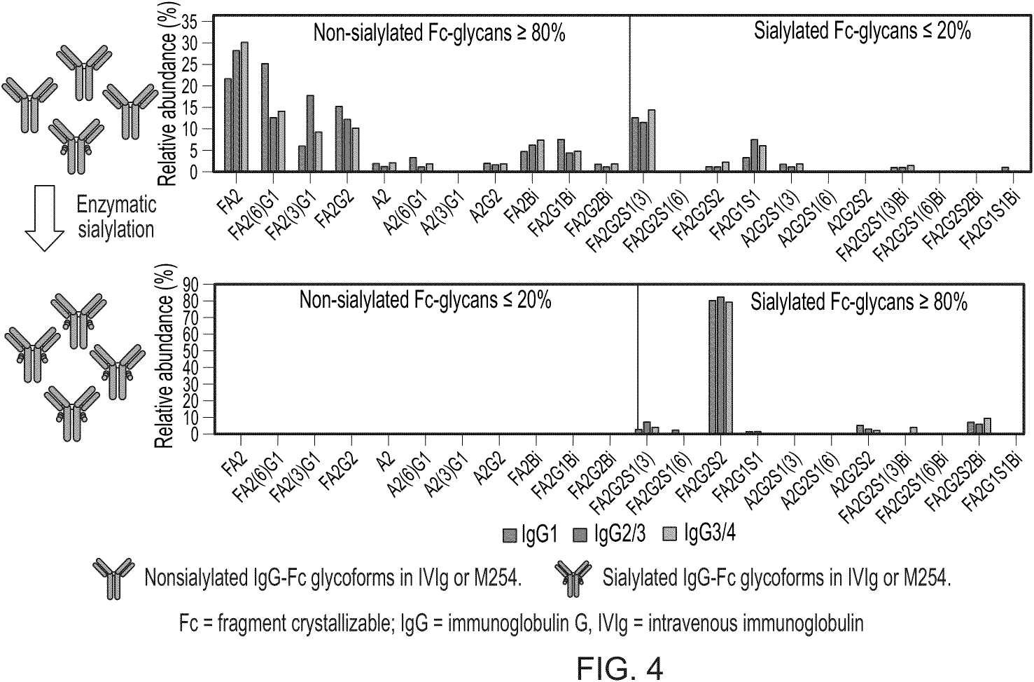

A representative example of the IgG-Fc glycan profile for such a reaction starting with IVIg and the reaction product is shown in the FIG. 4. In FIG. 4, the left panel is a schematic representation of enzymatic sialylation reaction to transform IgG to hsIgG; the right panel is the IgG Fc glycan profile for the starting IVIg and hsIgG. In this study, glycan profiles for the different IgG subclasses are derived via glycopeptide mass spectrometry analysis. The peptide sequences used to quantify glycopeptides for different IgG subclasses were: IgG1= EEQYNSTYR (SEQ ID NO: 1), IgG2/3 EEQFNSTFR (SEQ ID NO: 2), IgG3/4 EEQYNSTFR (SEQ ID NO: 3) and EEQFNSTYR (SEQ ID NO: 4).

The glycan data is shown per IgG subclass. Glycans from IgG3 and IgG4 subclasses cannot be quantified separately. As shown, for IVIg the sum of all the nonsialylated glycans is more than 80% and the sum of all sialylated glycans is < 20%. For the reaction product, the sum for all nonsialylated glycans is < 20% and the sum for all sialylated glycans is more than 80%. Nomenclature for different glycans listed in the glycoprofile use the Oxford notation for N linked glycans.

### Example 2: Alternative Sialylation Condition

An alternative suitable reaction conditions for galactosylation and sialylation to create hsIgG in 50 mM BIS-TRIS pH 6.9 include: galactosylation of IgG antibodies (e.g., pooled IgG antibodies, pooled immunoglobulins or IVIg) are as follows: 7.4 mM MnCl₂; 38 µmol UDP-Gal/g IgG antibody; and 7.5 units B4GalT/g IgG antibody with 16-24 hours of incubation at 37° C. followed by sialylation in 7.4 mM MnC12; 220 µmol CMP-NANA/g IgG antibody (added twice: half at the start of the reaction and half after 9-10 hrs); and 15 units ST6-Gal1/g IgG antibody with 30-33 hours of incubation at 37° C. The reaction can be carried out by adding the ST6-Gal1 and CMP-NANA to the galactosylation reaction. Alternatively, all of the reactants can be combined at the outset and the CMP-NANA supplemented.

### Example 3: Purification of hsIgG

It is desirable to have an efficient, economical method for purifying hsIgG prepared, for example, by any of the methods described herein. In order to prepared hsIgG suitable for use in a drug product is desirable to efficiently remove the enzymes, sugar nucleotide and manganese in the reaction(s) used to prepare hsIgG. Moreover, it can be desirable to reduce or eliminate the need for buffer exchange. Finally, since hsIgG can include a variety of IgG subclasses (e.g., IgG1, IgG2, etc.) it can be desirable to employ methods that produce a purified produce that does not unduly alter the subclass distribution of the unpurified material.

Protein A column chromatography was examined. Wash conditions including simple (NaCl, Na₂SO₄) and chaotropic (arginine, MgCl₂, KSCN) salts, buffer (phosphate, TRIS, BIS-TRIS, acetate, formate, glycolate, citrate, MOPS), organic modifier (ethylene glycol, propylene glycol), additives (imidazole, methyl galactopyranoside, lactose), and pH were examined.

Using A Mabselect PrismA column (GE Healthcare Life Sciences), loading of filtered reaction mixture (preferably in 50 mM MOPS at pH 7.4) followed by washing with 50 mM sodium acetate pH 4.99 and elution with glycine pH 3.0, produced hsIgG with a subclass profile similar to the initially loaded material (Loaded: IgG168%; IgG2 30%; IgG3 1.8% and IgG4 1.4%; Eluate: IgG170%; IgG2 29%; IgG3 0.9% and IgG4 1.1%). This column step substantially removed ST6-Gal. The Protein A column step was followed by a polishing step employing a Blue Trisacryl M (Pall Corp.) column in flow-thru mode. Here the loading buffer was 0.4 M NaCl, 50 mM Na citrate pH 4.5 and the wash buffer was 2 M NaCl, 50 mM sodium phosphate pH 7.0. This provided a very clean product. A different type of blue dye column with a sepharose base resin (HiTrap Blue HP; GE Healthcare Life Sciences) was used in a bind and elute mode. Here, loading in 50 mM sodium citrate pH 4.5 and elution in 1.5 M KCI, 32.5 mM sodium citrate, 35% ethylene glycol, pH 4.51 provided a very clean product.

### Example 4: Purification Process and Improvements

Various improvements to the following hsIgG manufacturing process show in FIG. 5 and described below are described in Examples 5-7.

After sialylation (step 4 in FIG. 5), the process comprises dilution of the reaction material 1:1 with 5x PBS to quench the reaction. The quenched reaction mixture is depth filtered to remove particulates (step 5 in FIG. 5) and is buffer exchanged into 50 mM tris, 100 mM NaCl, 10 mM EDTA pH 8.0 buffer for six diavolumes followed by buffer exchange into 250 mM glycine pH 5.0 (step 6 in FIG. 5). This buffer exchange step is to remove reaction components like nucleotides and manganese chloride. In addition, the EDTA buffer is a chelating agent that should also remove manganese chloride from solution.

In the glycine buffer, the pH of the process material is lowered to 3.5 for viral inactivation. It is held at the low pH for 90 minutes before being quenched and the pH raised to 4.5 (step 7 in FIG. 5). The quenched viral inactivation pool is diluted down to 4.5 mg/mL with 50 mM citrate, 400 mM NaCl pH 4.5 before being passed through a depth filter/nano filter combination (step 8 in FIG. 5). The filtrate is then buffer exchanged into 50 mM citrate, 400 mM NaCl pH 4.5 for 6 diavolumes and concentrated to 30 mg/mL (step 9 in FIG. 5). The final material is depth filtered one last time before the chromatography purification steps (step 10 in FIG. 5).

The first purification step (step 11 in FIG. 5) uses the strong cation exchange resin, Poros™ XS in the flow through mode. That means the material is directly loaded onto the column and the product is collected in the effluent while the impurities (ST6, B4GalT) stick to the resin. The flow through is then loaded onto a trisacryl blue (TAB) column that is also operated in flow through mode (step 12 in FIG. 5). The TAB resin also has significant capacity to remove reaction enzymes.

### Example 5: Strong Cation Exchange Resin in Bind and Elute Mode

The process comprises two TFF steps (buffer exchanges/one concentration), three depth filtration steps, and two dilutions before chromatography. These steps are expensive (material / buffer / operating time / lost product). Reducing the number of steps before the chromatography purification reduces cost, operation time, and the amount of material lost while still removing reaction impurities (e.g., nucleotides, enzymes, manganese chloride).

As described herein, steps 5-10 in FIG. 5 can be eliminated from the process by diluting the quenched (5x PBS) reaction mixture with 50 mM citrate pH 4.5 and using the strong cation exchange resin, Poros™ XS, in the bind and elute mode. The reaction mixture will bind to the Poros™ XS strong cation exchange resin while the nucleotides and manganese chloride flow through into the waste. From here, the bound material can be selectively eluted using the same 50 mM citrate, 400 mM NaCl pH 4.5 solution used in the current process. The enzymes, requiring a higher salt concentration for elution, will remain bound to the column. In addition, the eluted product is now in the same buffer that is required for the TAB resin purification.

### Resin Screening

Five different strong cation exchange resins were evaluated for their ability to separate IVIg from ST6 spiked into solution: Poros™ XS, Capto™ S, Capto™ SP Impres, SP HP, and Capto™ S Impact. The load material was prepared by diluting 0.55 mL IVIg at 100 mg/mL 1:10 with 50 mM citrate pH 4.5 and then spiking in 3.3 mL of ST6 at 16 mg/mL. For each resin, 8 mL of the load material was applied to the column and then eluted by linear gradient into 50 mM citrate, 1 M NaCl pH 4.5. The ability of each resin to separate IVIg and ST6 was evaluated by observing the chromatograms and by using Unicorn to calculate the peak resolution shown in the equation below (t is mean retention time, W is peak width). The resolution calculations (Table 9) showed that Poros™ XS performed the best for separation of IVIg from ST6.

$${R}_{S} = \frac{{t}_{b} - {t}_{a}}{2 \left({W}_{a} + {W}_{b}\right)}$$

TABLE 9

Resolution of resins

| Resin | Resolution |
|---|---|
| Capto™ S Impact | 0.87 |
| Capto™ S | 0.37 |
| Capto™ SP Impres | 0.90 |
| SP HP | 1.01 |
| Poros™ XS | 1.17 |

### Binding Capacity Determination

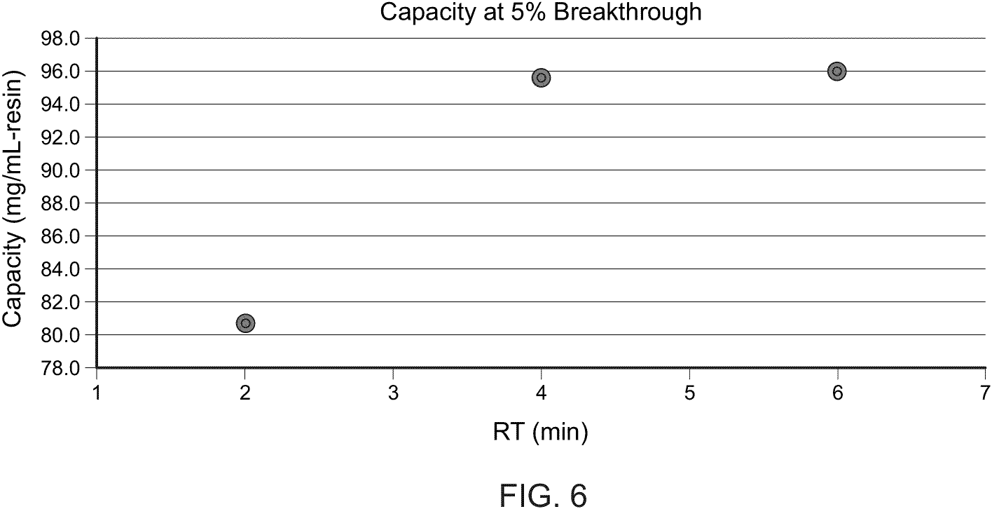

Operating the Poros™ resin in flow through mode is advantageous because it allows for only one cycle and a smaller resin requirement since the resin only needs to bind the impurities. When using the Poros™ XS resin in bind and elute mode the number of cycles required for complete processing depends on how much product it can bind at one time. A load solution was made by diluting IVIg 1:20 with 50 mM citrate pH 4.5. The load solution was applied to the column at 2-, 4- and 6-minute residence times and the binding capacity evaluated at a 5% breakthrough. FIG. 6 shows that not much capacity is gained by operating above a 4 minute residence time, lower than the 5-minute residence time typical for chromatography operations. In addition, the capacity is very high at ~96 mg/mL resin.

### Yield & Residual ST6 Evaluation

The yield and residual ST6 for differing load capacity and elution buffer conductivity was evaluated over a number of runs to identify the best elution conditions and loading capacity (Table 10). The loading solution was prepared by diluting 2.8 g of IVIg, and 500 uL of ST6 at 15.6 mg/mL and diluting the solution 1:20 with 50 mM citrate pH 4.5. All steps were operated at a 5-minute residence time. Load material is 2,700 ppm in ST6.

TABLE 10

| Run | Load Capacity (mg/mL-resin) | Elution Conductivity (mS/cm) | Load Mass (mg) | Eluate Vol (mL) | Eluate Cone (mg/mL) | Yield (%) | ST6 (ppm) |
|---|---|---|---|---|---|---|---|
| 1 | 55 | 44 | 110 | 9 | 11.2 | 91.6 | 19.9 |
| 2 | 55 | 49 | 110 | 9 | 11.7 | 95.7 | 71.2 |
| 3 | 70 | 44 | 140 | 9 | 14.5 | 93.2 | 9.2 |
| 4 | 70 | 49 | 140 | 9 | 14.5 | 93.2 | 73.5 |
| 5 | 85 | 44 | 170 | 9 | 17.1 | 90.5 | 15.8 |
| 6 | 85 | 49 | 170 | 9 | 17.1 | 90.5 | 84.3 |
| 7 | 55 | 39.4 | 110 | 9 | 11.1 | 90.8 | 6.1 |
| 8 | 70 | 39.4 | 140 | 9 | 13.6 | 87.4 | 8.2 |
| 9 | 85 | 39.4 | 170 | 9 | 16.9 | 89.5 | 6.7 |

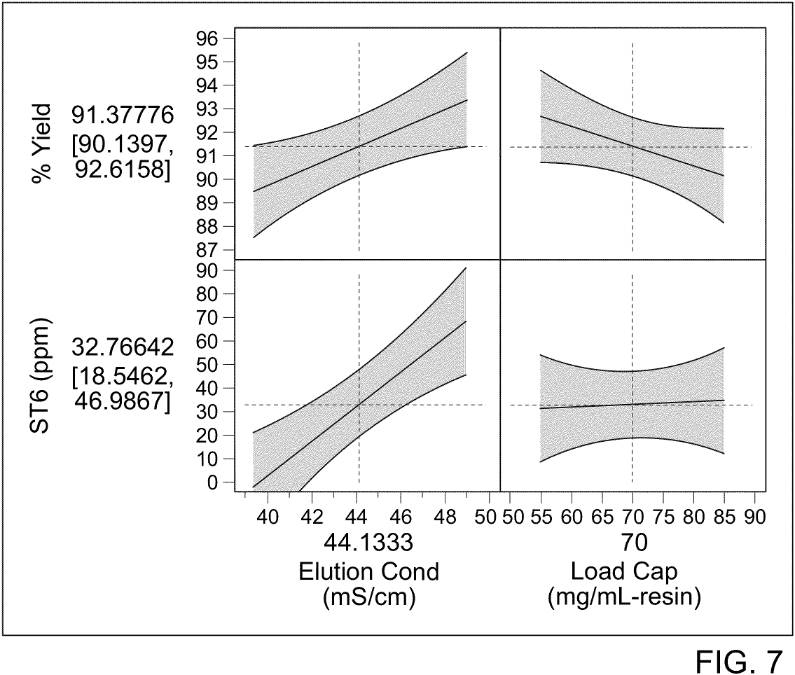

Statistical analysis showed that the elution conductivity was the only significant parameter in the regression models for both yield and residual ST6. The prediction profiler in FIG. 7 shows that increasing load capacity did not have a statistically significant effect on yield.

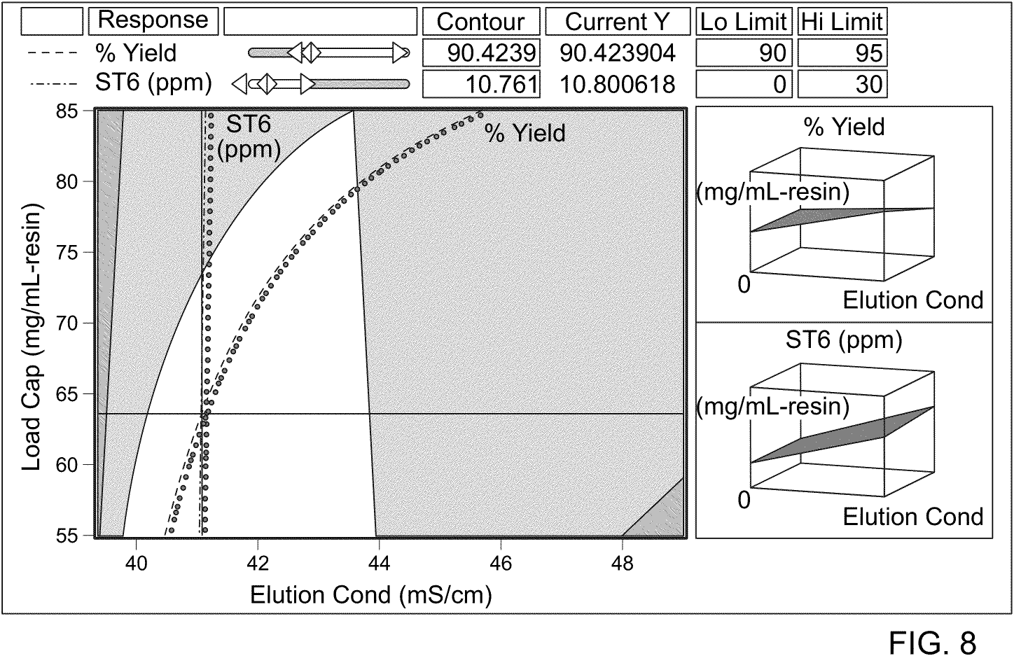

FIG. 8 shows the operating range for a yield between 90- 95% and residual ST6 levels less than 30 ppm. The white space shows the operating range which suggest that elution conductivities between 39-44 mS/cm can be used for loading capacities less than 55 mg/mL-resin. As the loading capacity increases, this range becomes narrower. These results essentially confirm that the 50 mM citrate, 400 mM NaCl pH 4.5 (400 mM NaCl corresponds to 40 mS/cm conductivity) is the best option for the elution buffer.

### Purification of hsIVIg Reaction Mixture

A number of runs were performed with a hsIVIg generated using the sialylation procedure developed in MOPS (Table 11). The hsIVIg reaction mixture was quenched with 5x PBS with a 1:1 dilution and then diluted more with 50 mM citrate pH 4.5. 50 mM citrate, 400 mM NaCl pH 4.5 was used as elution buffer for all the runs below. Concentrations of 7.15 mg/mL correspond to 1:20 dilution with 50 mM citrate pH 4.5. Five minute residence times were used for all steps.

TABLE 11

| Run | Load Capacity (mg/mL-resin) | Load Conc. (mg/mL) | Yield % | ST6 (ppm) |
|---|---|---|---|---|
| 3 | 28.6 | 14.3 | 87.5 | 31.9 |
| 4 | 57.2 | 7.15 | 91.9 | 33.3 |
| 5 | 14.3 | 7.15 | 97.3 | 38.4 |
| 6 | 28.6 | 7.15 | 91.4 | 29.1 |
| 10 | 35.7 | 6.08 | 93.5 | 24.1 |

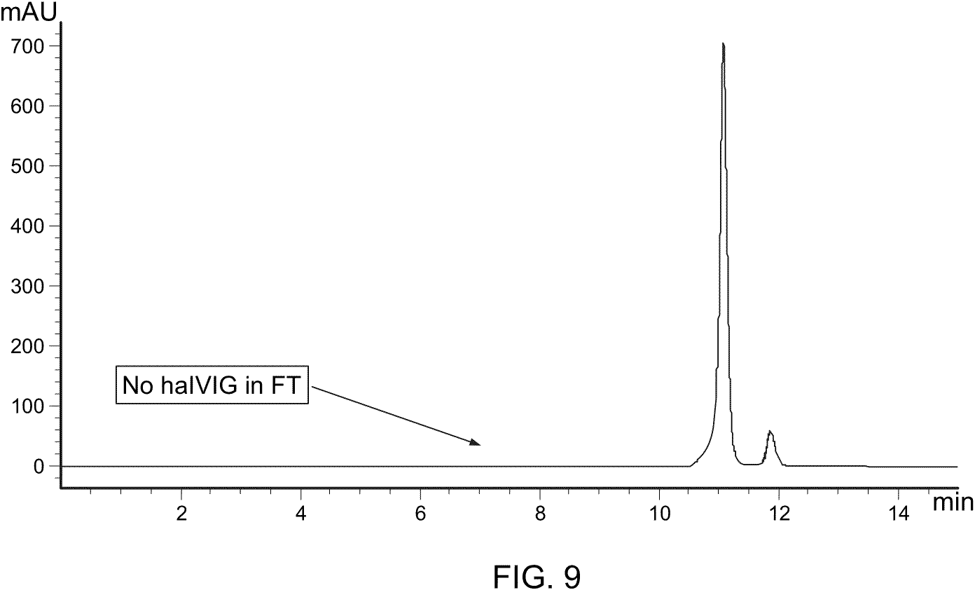

The flow through step contains no IgG only nucleotides and manganese chloride as seen by the SEC chromatogram of the flow through in FIG. 9.

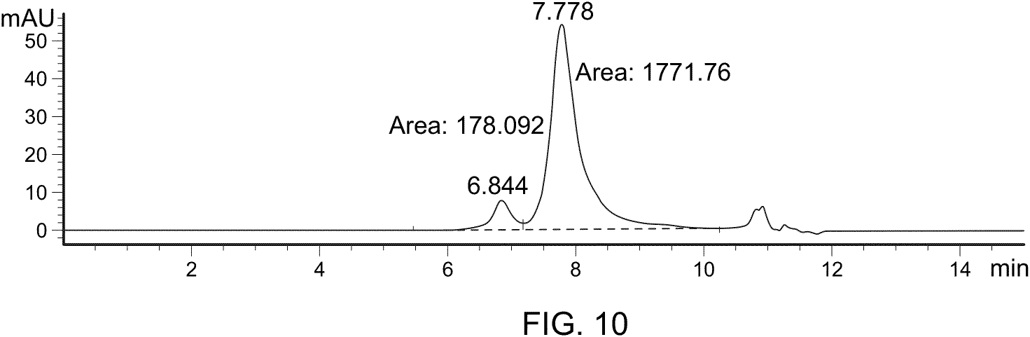

This can be compared to the SEC of the eluate in FIG. 10, which shows a dimer peak at 6.8 minutes retention and the monomer peak at 7.8 minutes retention.

### Results Summary

These results show that by diluting the reaction mixture directly into 50 mM citrate pH 4.5 and loading onto the same Poros™ XS resin, the current hsIVIg process is improved significantly. Binding the hsIVIg to the resin allows for a much simpler path to removing nucleotides and manganese chloride (achieved by buffer exchange previously). Using 50 mM citrate, 400 mM NaCl pH 4.5, it is clear that significant ST6 enzyme clearance can be achieved. In addition, the Poros™ XS eluate pool is in the appropriate conditions for the subsequent TAB purification step.

In a manufacturing scale process, a single depth filtration step may still be used. The sialylation reaction mixture is often very turbid and not suitable for direct loading to a chromatography resin. So, after diluting the quenched reaction mixture with 50 mM citrate pH 4.5, the process material may be depth filtered before loading onto the Poros™ XS resin.

### Example 6: Alternative Columns

The current manufacturing process of M254 uses a trisacryl blue (TAB) column in flow-through mode which does not withstand high concentration of sodium hydroxide wash. This results in a higher level of bioburden in some batches. Different blue resins have been evaluated and Capto™ Blue High Sub (Capto™ Blue HS) has been found to be a good alternative for the TAB column with similar recovery and better enzymes clearance. In addition, Capto™ Blue High Sub can withstand a high sodium hydroxide wash and has the potential to reduce the number of steps of the current purification process.

### Evaluation of Capto™ Blue and Blue Sepharose™

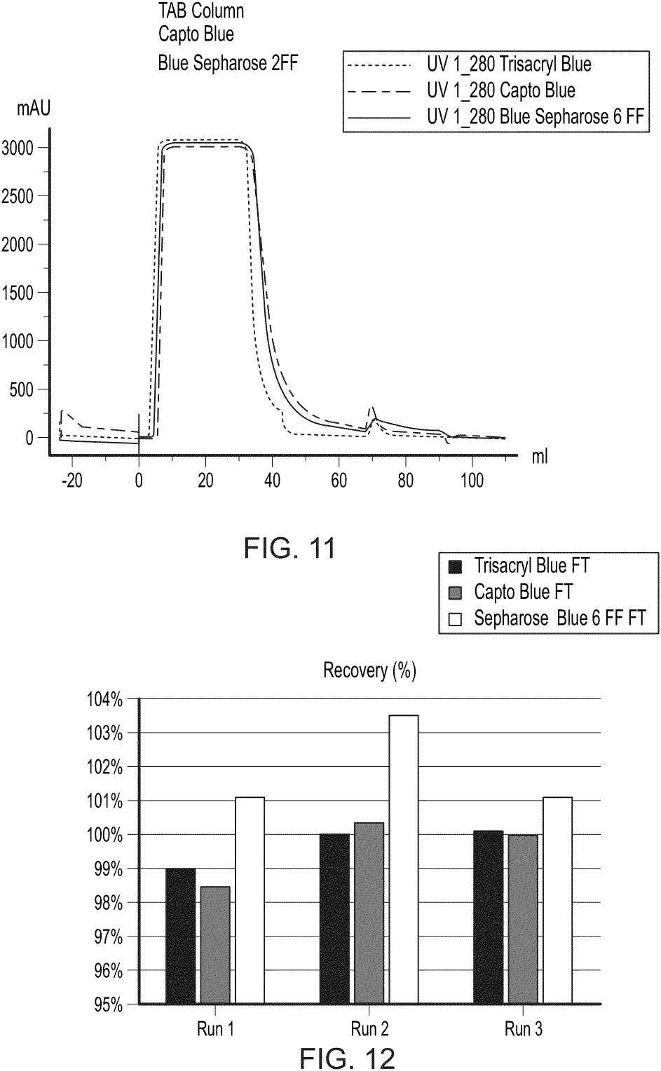

Two chromatography resins have been evaluated against TAB to determine if the recovery is impacted. For that purpose, 250 mg of IVIg diluted 1: 1 in 800 mM NaCL +100 mM citrate pH 4.5 and loaded directly in triplicate. Overlay of the chromatograms of one of the runs is shown in FIG. 11 while the recovery of the three runs is reported in FIG. 12. As can be seen, the recovery (99%) is not impacted.

### Comparison of Capto™ Blue and Capto™ Blue HS

Capto™ Blue and Capto™ Blue HS have been compared to evaluate the recovery and enzyme clearance. Multiple runs were executed where either hsIGIV (post strong cation exchange column flow-through material) was loaded directly or loaded after dilution. Table 12 summarizes different load conditions while Table 13 is showing the mass balance:

TABLE 12

Loading conditions

| | | | | |
|---|---|---|---|---|
| | Run #1 | Run # 2 | Run #3 | Run #4 |
| Chromatography Resin | 1 mL Capto™ Blue | 1 mL Capto™ Blue | 1 mL Capto™ Blue HS | 1 mL Capto™ Blue HS |
| Starting material | hsIVIg SCX FT | hsIVIg SCX Load | hsIVIg SCX FT | *hsIVIg SCX FT diluted (1:1) |
| Protein Concentration (mg/mL) | 25.2 | 25.9 | 25.2 | 12.7 |
| Amount loaded (g) | 2 | 2 | 1.1 | 1.1 |
| IVIg g/mL of resins | 2 | 2 | 1.1 | 1.1 |
| Flow rate (mL/min) | 0.17 | 0.17 | 0.17 | 0.17 |
| Residence time (min) | 6 | 6 | 6 | 6 |
| Spiked enzymes /mg of IVIg | N/A | N/A | 0.2 uL/mg | N/A |

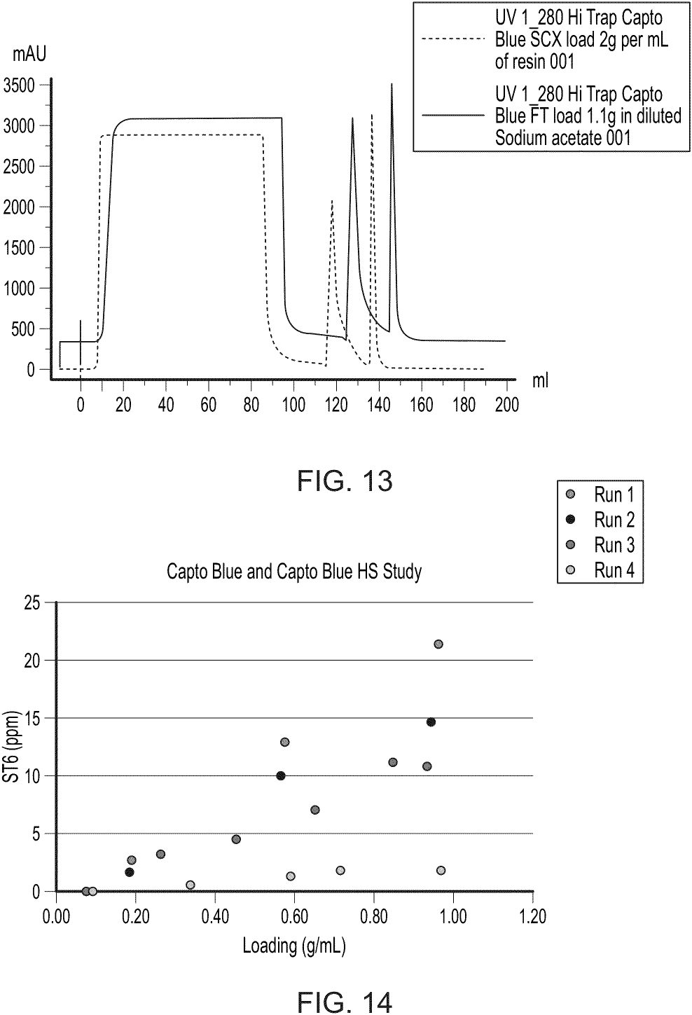

As shown in FIG. 13, overlay chromatograms of run 1 and 4 look very similar.

TABLE 13

Recovery of Capto™ B and Capto™ Blue HS

| Capto™ Blue run | Mass Balance (%) | Strip (%) |
|---|---|---|
| Run 1 (Capto™ B) | 97.5 | 2.2 |
| Run2 (Capto™ B) | 96.7 | 2.2 |
| Run 3 (Capto™ B HS) | 99.6 | 4.9 |
| Run 4 (Capto™ B HS) | 96.2 | 6.7 |

Recovery of the Capto™ Blue HS is very similar to the Capto™ blue and 96% recovery is considered satisfactory to be implemented instead of TAB column.

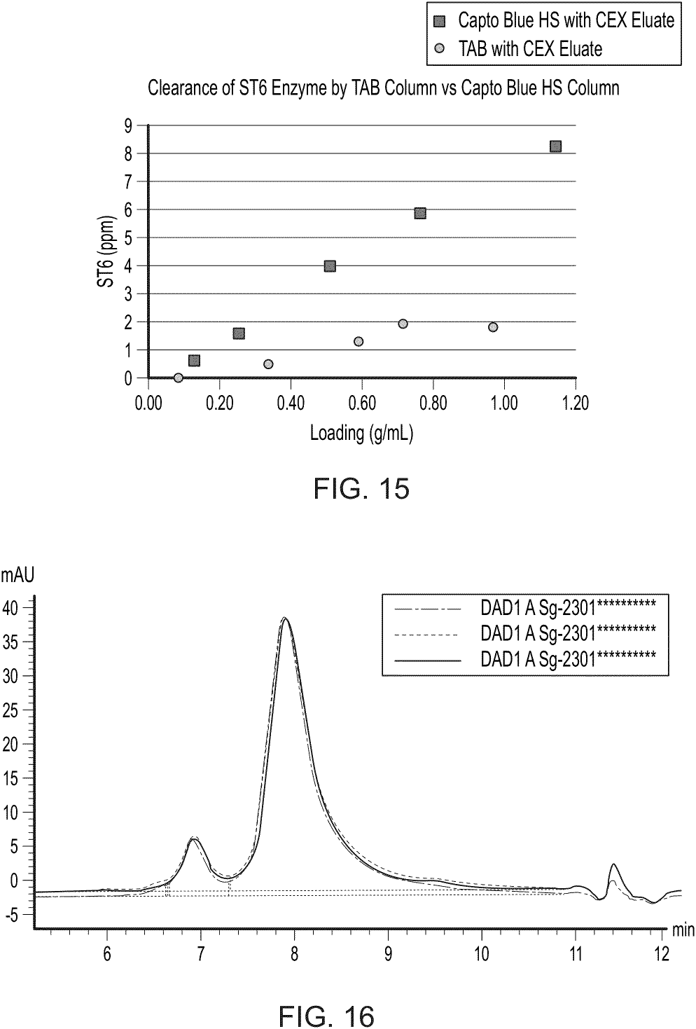

Residual ST6 after Capto™ Blue and Capto™ Blue HS was also evaluated and found to be as low if not lower than TAB as shown in FIG. 14 and FIG. 15.

### Impact of Using Capto™ Blue HS on Product Attributes

IVIg from Capto™ Blue HS flow through and eluate were compared to the load to evaluate impact on the product quality. Analytical SEC of the load, flow-through and eluate were compared; no differences were found, as demonstrated in FIG. 16 and Table 14.

TABLE 14

Level of aggregate, dimer and monomer are very similar

| | RT of the monomer (min) | Agg.(%) | Dimer (%) | Monomer (%) | LMW (%) |
|---|---|---|---|---|---|
| Load | 7.889 | 1.2 | 9.9 | 84.0 | 4.9 |
| FT | 7.905 | 1.5 | 10.0 | 83.7 | 4.8 |
| Eluate | 7.903 | 1.3 | 9.4 | 84.6 | 4.7 |

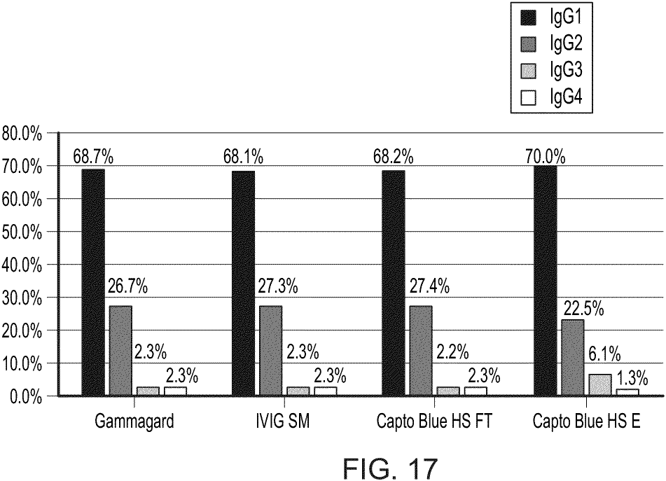

IgG subclass distribution was evaluated by mass spectrometry (FIG. 17). Analysis before and after Capto™ Blue High Sub in flow through mode was compared for two sources of IVIg and not found to change.

### Example 7: Elimination of Cation Exchange Column

### Capto™ Blue HS

In order to reduce the number of current manufacturing process steps, different conditions were evaluated and residual ST6 was evaluated:
Run1: Capto™ Blue HS flow through of an experiment with residual ST6 of 651 ppm was used as loading test material to evaluate 5X PBS and MOPS at pH 4.5 buffer as loading buffer which will mimic loading material directly into the column after the reaction.
Run 2: Post viral inactivation material with residual ST6 of 1400 ppm was loaded directly onto the Capto™ Blue HS resin using 250 mM Glycine pH 4.5.
Run 3: Post viral inactivation material with residual ST6 of 1400 ppm was loaded directly onto the Capto™ Blue HS resin using 250 mM Glycine + 100 mM Sodium Chloride pH 4.5.

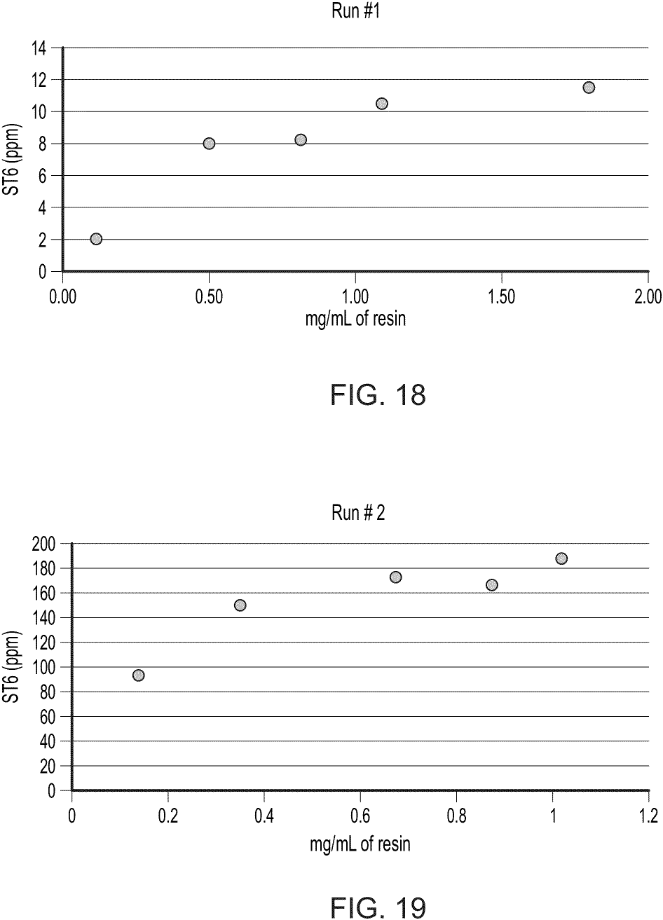

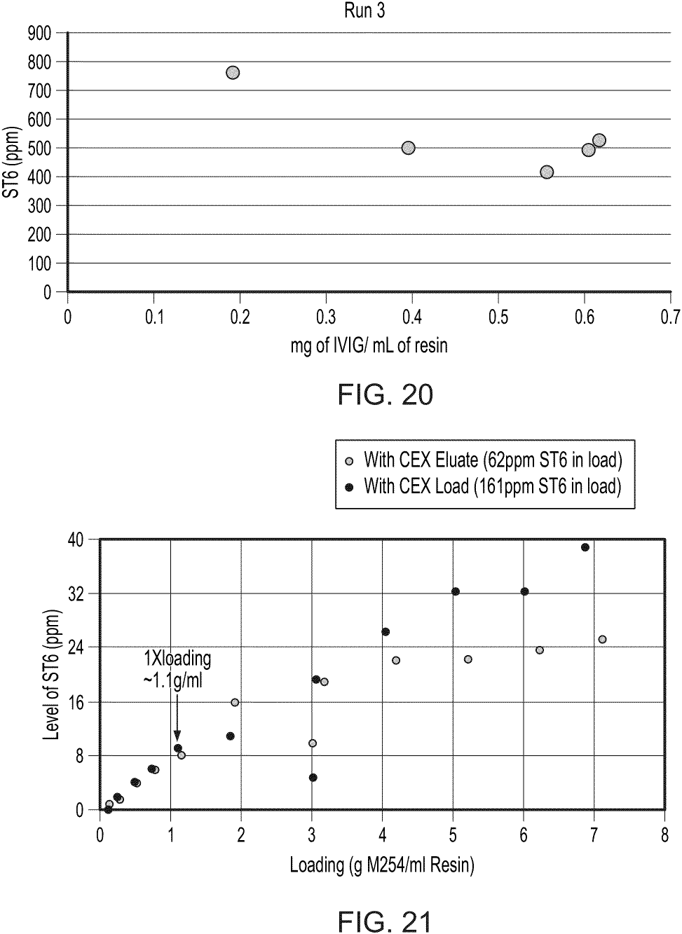

The residual ST6 of the three runs were evaluated, as shown in FIG. 18 (run 1), FIG. 19 (run 2), and FIG. 20 (run 3). Loading the reaction mixture directly onto the Capto™ Blue High Sub can significantly reduce the level of ST6. However, increasing the salt even with 100 mM NaCl has a negative impact on residual ST6.

### Trisacryl Blue (TAB)

The current manufacturing process has two chromatography columns, CEX followed by TAB to reduce the level of added ST6 during enzymatic reaction. Residual level of ST6 was 2.5-7.2 ppm for the historical manufactured drug substance lots. Maximum loading for the historical manufacturing process using the TAB column was 1.1 g IVIg/ml resin (1X).

In the present study, TAB column runs were performed to understand the feasibility to increase the loading capacity and to eliminate CEX column from the current manufacturing process.

Pre-packed 1ml TAB columns were used for the loading study. TAB column loading study runs were performed with CEX load and CEX eluate obtained from 1.5 Kg GMP run lot # 20006. TAB column runs were performed by using AKTA Avant 150. Load flow rate was maintained at 0.17 ml/min to achieve residence time similarly to manufacturing column operation. Load flow-through fractions were collected, protein concentration measured by A280, and residual level of ST6 determined by ELISA.

As shown in FIG. 21, level of residual ST6 was 0.1 - 9 ppm for the TAB column flow-through fractions when loading 0.1 - 1.1 g/ml where the x-axis is the cumulative protein flow-through. If these flow-through fractions were pooled then it is reasonable to estimate the level of ST6 around 5 ppm (with 1.1 g/ml loading), in other words similar to the historical manufacturing drug substance lots. Results from the present study confirm that maximum loading should be kept <1.1 g/ml for the TAB column to maintain the ST6 levels seen in DS lots. Level of ST6 values were 161 ppm and 62 ppm for the manufacturing scale CEX load and CEX eluate indicated CEX column in the current manufacturing process reduce the level of ST6.

In the present study, TAB column run was performed with CEX load, in other words without CEX column purification. As shown in the FIG. 21, residual level of ST6 values were similar for the TAB column flow-through fractions (0.1-9 ppm) with two load materials, CEX load (without CEX column purification, 161 ppm) and CEX eluate (with CEX column as in the current manufacturing process, 62 ppm).

Results in the present study indicated 1.1 g/ml as maximum loading for the TAB column to decrease the level of ST6 ≤7.2 ppm for drug substance similarly to historical manufacturing runs (ST6 level 2.5-7.2 ppm). Additionally, results in the present study suggest without CEX column, TAB column efficiently decreases the level of ST6 to meet the current target (drug substance ≤7.2 ppm).

### Example 8: Ammonium Sulfate Precipitation

IVIg is isolated and purified from human plasma in some methods by precipitation using either ethanol or polyethylene glycol at low temperature. Here, we evaluate precipitation of hsIVIg using ammonium sulfate to remove residual ST6.

For that purpose, IVIg and hsIVIg were precipitated as follows. A saturated solution of ammonium sulfate was prepared by adding the powder directly into a 100 mL of water while stirring. Once a significant amount of the powder was precipitated the solution was left stirring for 30 min then decanted from the precipitate and added as is to the IVIG solution. The saturated ammonium sulfate solution was added until the solution of the IVIG turned white and no change in the color was seen. The precipitate was collected by centrifugation or by sterile filtration.

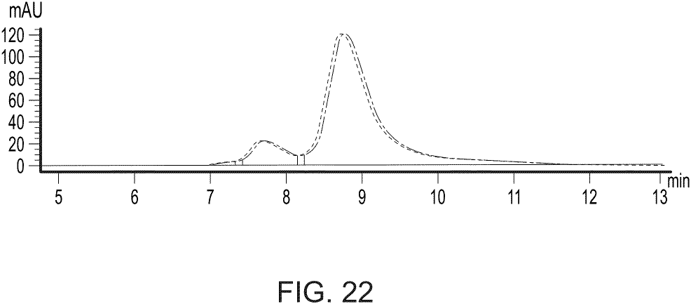

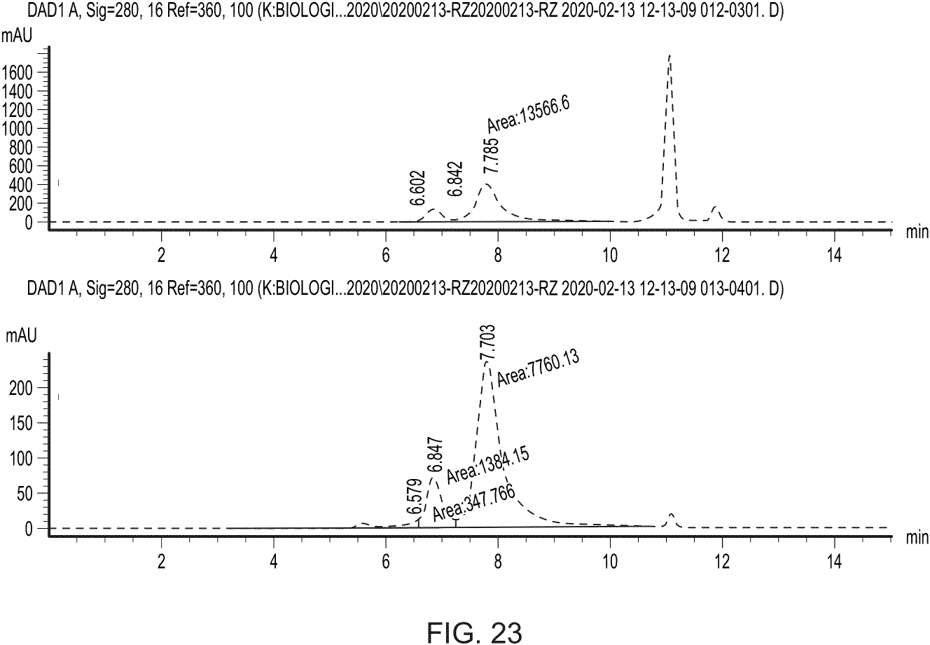

FIG. 22 shows an overlay of the precipitated IVIg versus the starting IVIg and the precipitated material and FIG. 23 shows hsIVIg before and after precipitation using sterile filtration as a way of collecting the precipitated material. One can clearly see the removal of nucleotides which elute at ~11 min. As shown in Table 15, there was also a significant reduction of residual ST6 enzymes.

From a single experiment, we can see that it is possible to remove residual enzymes by precipitation. Aggregate seen when material was collected by filtration is not an issue knowing that filtration is not a way to collect IVIg.

TABLE 15

Ammonium Sulfate Precipitation Results

| Mode | Starting material | Recovery (%) | ST6 (ppm) | Aggregates by SEC (%) | Dimer by SEC (%) | Monomer by SEC (%) |
|---|---|---|---|---|---|---|
| Centrifugation | IVIg | 86 | N/A | 1.4 | 9.8 | 88.8 |
| Filtration | hs-IVIg | 97 | 36 | 3.7 | 14.6 | 81.8 |

### SEQUENCES

SEQ ID NO: 1 (IgG1)
EEQYNSTYR

SEQ ID NO: 2 (IgG2/3)
EEQFNSTFR

SEQ ID NO: 3 (IgG3/4)
EEQYNSTFR

SEQ ID NO: 4 (IgG3/4)
EEQFNSTYR

SEQ ID NO: 5 (NP_001488.2 B4GALT1 [organism=Homo sapiens] [GeneID=2683]
[isoform=1])
MRLREPLLSGSAAMPGASLQRACRLLVAVCALHLGVTLVYYLAGRDLSRL
PQLVGVSTPLQGGSNSAAAIGQSSGELRTGGARPPPPLGASSQPRPGGDS
SPVVDSGPGPASNLTSVPVPHTTALSLPACPEESPLLVGPMLIEFNMPVD
LELVAKQNPNVKMGGRYAPRDCVSPHKVAIIIPFRNRQEHLKYWLYYLHP
VLQRQQLDYGIYVINQAGDTIFNRAKLLNVGFQEALKDYDYTCFVFSDVD
LIPMNDHNAYRCFSQPRHISVAMDKFGFSLPYVQYFGGVSALSKQQFLTI
NGFPNNYWGWGGEDDDIFNRLVFRGMSISRPNAVVGRCRMIRHSRDKKNE
PNPQRFDRIAHTKETMLSDGLNSLTYQVLDVQRYPLYTQITVDIGTPS

SEQ ID NO: 6 (NP_001365424.1 B4GALT1 [organism=Homo sapiens] [GeneID=2683]
[isoform=2])
MPGASLQRACRLLVAVCALHLGVTLVYYLAGRDLSRLPQLVGVSTPLQGG
SNSAAAIGQSSGELRTGGARPPPPLGASSQPRPGGDSSPVVDSGPGPASN
LTSVPVPHTTALSLPACPEESPLLVGPMLIEFNMPVDLELVAKQNPNVKM
GGRYAPRDCVSPHKVAIIIPFRNRQEHLKYWLYYLHPVLQRQQLDYGIYV
INQAGDTIFNRAKLLNVGFQEALKDYDYTCFVFSDVDLIPMNDHNAYRCF
SQPRHISVAMDKFGFSLPYVQYFGGVSALSKQQFLTINGFPNNYWGWGGE
DDDIFNRLVFRGMSISRPNAVVGRCRMIRHSRDKKNEPNPQRFDRIAHTK
ETMLSDGLNSLTYQVLDVQRYPLYTQITVDIGTPS

SEQ ID NO: 7 (NP_001365425.1 B4GALT1 [organism=Homo sapiens] [GeneID=2683]
[isoform=3])
MRLREPLLSGSAAMPGASLQRACRLLVAVCALHLGVTLVYYLAGRDLSRL
PQLVGVSTPLQGGSNSAAAIGQSSGELRTGGARPPPPLGASSQPRPGGDS
SPVVDSGPGPASNLTSVPVPHTTALSLPACPEESPLLVGPMLIEFNMPVD
LELVAKQNPNVKMGGRYAPRDCVSPHKVAIIIPFRNRQEHLKYWLYYLHP
VLQRQQLDYGIYVINQAGDTIFNRAKLLNVGFQEALKDYDYTCFVFSDVD
LIPMNDHNAYRCFSQPRHISVAMDKFGFRLVFRGMSISRPNAVVGRCRMI
RHSRDKKNEPNPQRFDRIAHTKETMLSDGLNSLTYQVLDVQRYPLYTQIT
VDIGTPS

SEQ ID NO: 8 (NP_001365426.1 B4GALT1 [organism=Homo sapiens] [GeneID=2683]
[isoform=4])
MRLREPLLSGSAAMPGASLQRACRLLVAVCALHLGVTLVYYLAGRDLSRL
PQLVGVSTPLQGGSNSAAAIGQSSGELRTGGARPPPPLGASSQPRPGGDS
SPVVDSGPGPASNLTSVPVPHTTALSLPACPEESPLLVGPMLIEFNMPVD
LELVAKQNPNVKMGGRYAPRDCVSPHKVAIIIPFRNRQEHLKYWLYYLHP
VLQRQQLDYGIYVINQYEKIRRLLW

SEQ ID NO: 9
MRLREPLLSGSAAMPGASLQRACR

SEQ ID NO: 10
LLVAVCALHLGVTLVYYLAG

SEQ ID NO: 11
RDLSRLPQLVGVSTPLQGGSNSAAAIGQSSGELRTGGARPPPPLGASSQP
RPGGDSSPVVDSGPGPASNLTSVPVPHTTALSLPACPEESPLLVGPMLIE
FNMPVDLELVAKQNPNVKMGGRYAPRDCVSPHKVAIIIPFRNRQEHLKYW
LYYLHPVLQRQQLDYGIYVINQAGDTIFNRAKLLNVGFQEALKDYDYTCF
VFSDVDLIPMNDHNAYRCFSQPRHISVAMDKFGFSLPYVQYFGGVSALSK
QQFLTINGFPNNYWGWGGEDDDIFNRLVFRGMSISRPNAVVGRCRMIRHS
RDKKNEPNPQRFDRIAHTKETMLSDGLNSLTYQVLDVQRYPLYTQITVDI
GTPS

SEQ ID NO: 12 (B4GalT)
GPASNLTSVPVPHTTALSLPACPEESPLLVGPMLIEFNMPVDLELVAKQN
PNVKMGGRYAPRDCVSPHKVAIIIPFRNRQEHLKYWLYYLHPVLQRQQLD
YGIYVINQAGDTIFNRAKLLNVGFQEALKDYDYTCFVFSDVDLIPMNDHN
AYRCFSQPRHISVAMDKFGFSLPYVQYFGGVSALSKQQFLTINGFPNNYW
GWGGEDDDIFNRLVFRGMSISRPNAVVGRCRMIRHSRDKKNEPNPQRFDR
IAHTKETMLSDGLNSLTYQVLDVQRYPLYTQITVDIGTPS

SEQ ID NO: 13 (B4GalT)
gssplldmGPASNLTSVPVPHTTALSLPACPEESPLLVGPMLIEFNMPVD
LELVAKQNPNVKMGGRYAPRDCVSPHKVAIIIPFRNRQEHLKYWLYYLHP
VLQRQQLDYGIYVINQAGDTIFNRAKLLNVGFQEALKDYDYTCFVFSDVD
LIPMNDHNAYRCFSQPRHISVAMDKFGFSLPYVQYFGGVSALSKQQFLTI
NGFPNNYWGWGGEDDDIFNRLVFRGMSISRPNAVVGRCRMIRHSRDKKNE
PNPQRFDRIAHTKETMLSDGLNSLTYQVLDVQRYPLYTQITVDIGTPSpr
dhhhhhhh

SEQ ID NO: 14 (NP_001340845.1 (NP_003023.1, NP_775323.1) ST6GAL1 [organism=Homo
sapiens] [GeneID=6480] [isoform=a])
MIHTNLKKKFSCCVLVFLLFAVICVWKEKKKGSYYDSFKLQTKEFQVLKS
LGKLAMGSDSQSVSSSSTQDPHRGRQTLGSLRGLAKAKPEASFQVWNKDS
SSKNLIPRLQKIWKNYLSMNKYKVSYKGPGPGIKFSAEALRCHLRDHVNV
SMVEVTDFPFNTSEWEGYLPKESIRTKAGPWGRCAVVSSAGSLKSSQLGR
EIDDHDAVLRFNGAPTANFQQDVGTKTTIRLMNSQLVTTEKRFLKDSLYN
EGILIVWDPSVYHSDIPKWYQNPDYNFFNNYKTYRKLHPNQPFYILKPQM
PWELWDILQEISPEEIQPNPPSSGMLGIIIMMTLCDQVDIYEFLPSKRKT
DVCYYYQKFFDSACTMGAYHPLLYEKNLVKHLNQGTDEDIYLLGKATLPG
FRTIHC

SEQ ID NO: 15 (NP_775324.1 ST6GAL1 [organism=Homo sapiens] [GeneID=6480]
[isoform=b])
MNSQLVTTEKRFLKDSLYNEGILIVWDPSVYHSDIPKWYQNPDYNFFNNY
KTYRKLHPNQPFYILKPQMPWELWDILQEISPEEIQPNPPSSGMLGIIIM
MTLCDQVDIYEFLPSKRKTDVCYYYQKFFDSACTMGAYHPLLYEKNLVKH
LNQGTDEDIYLLGKATLPGFRTIHC

SEQ ID NO: 16
MIHTNLKKK

SEQ ID NO: 17
FSCCVLVFLLFAVICVW

SEQ ID NO: 18
KEKKKGSYYDSFKLQTKEFQVLKSLGKLAMGSDSQSVSSSSTQDPHRGRQ
TLGSLRGLAKAKPEASFQVWNKDSSSKNLIPRLQKIWKNYLSMNKYKVSY
KGPGPGIKFSAEALRCHLRDHVNVSMVEVTDFPFNTSEWEGYLPKESIRT
KAGPWGRCAVVSSAGSLKSSQLGREIDDHDAVLRFNGAPTANFQQDVGTK
TTIRLMNSQLVTTEKRFLKDSLYNEGILIVWDPSVYHSDIPKWYQNPDYN
FFNNYKTYRKLHPNQPFYILKPQMPWELWDILQEISPEEIQPNPPSSGML
GIIIMMTLCDQVDIYEFLPSKRKTDVCYYYQKFFDSACTMGAYHPLLYEK
NLVKHLNQGTDEDIYLLGKATLPGFRTIHC

SEQ ID NO: 19 (ST6Gal1)
AKPEASFQVWNKDSSSKNLIPRLQKIWKNYLSMNKYKVSYKGPGPGIKFS
AEALRCHLRDHVNVSMVEVTDFPFNTSEWEGYLPKESIRTKAGPWGRCAV
VSSAGSLKSSQLGREIDDHDAVLRFNGAPTANFQQDVGTKTTIRLMNSQL
VTTEKRFLKDSLYNEGILIVWDPSVYHSDIPKWYQNPDYNFFNNYKTYRK
LHPNQPFYILKPQMPWELWDILQEISPEEIQPNPPSSGMLGIIIMMTLCD
QVDIYEFLPSKRKTDVCYYYQKFFDSACTMGAYHPLLYEKNLVKHLNQGT
DEDIYLLGKATLPGFRTIHC

SEQ ID NO: 20 (ST6Gal1)
gssplldmlehhhhhhhhmAKPEASFQVWNKDSSSKNLIPRLQKIWKNYL
SMNKYKVSYKGPGPGIKFSAEALRCHLRDHVNVSMVEVTDFPFNTSEWEG
YLPKESIRTKAGPWGRCAVVSSAGSLKSSQLGREIDDHDAVLRFNGAPTA
NFQQDVGTKTTIRLMNSQLVTTEKRFLKDSLYNEGILIVWDPSVYHSDIP
KWYQNPDYNFFNNYKTYRKLHPNQPFYILKPQMPWELWDILQEISPEEIQ
PNPPSSGMLGIIIMMTLCDQVDIYEFLPSKRKTDVCYYYQKFFDSACTMG
AYHPLLYEKNLVKHLNQGTDEDIYLLGKATLPGFRTIHC

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of producing a purified hypersialylated IgG (hsIgG) composition with greater than 75% of the branched Fc glycans on the hsIgG have a sialic acid on both the a1,3 branch and the a1,6 branch, the method comprising:
(a) providing a hsIgG composition comprising hsIgG and ST6Gal or an enzymatically active portion thereof;
(b) diluting the composition in a citrate buffer at about 50 mM, about pH 4.5, thereby producing a buffered antibody composition;
(c) applying the buffered antibody composition to a chromatography column comprising a resin with a sulfonic acid functional group under conditions that bind the hsIgG as well as the ST6Gal or enzymatically active portion thereof; and
(d) selectively eluting the IgG from the column, thereby producing a purified hsIgG composition with greater than 75% of the branched Fc glycans on the hsIgG have a sialic acid on both the a1,3 branch and the a1,6 branch.

2. (canceled)

3. The method of claim 1 or claim 2, wherein the CEX column comprises a resin having a SO₃^{—} functional group.

4. The method of claim 1 , wherein selectively eluting the hsIgG comprises eluting in a buffer comprising about 400 mM or more NaCl.

5-16. (canceled)

17. A method of producing a purified hypersialylated IgG (hsIgG) composition, the method comprising:
(a) providing a hsIgG composition comprising hsIgG and ST6Gal or an enzymatically active portion thereof;
(b) diluting the composition in a buffer suitable for use with the column, thereby producing a buffered antibody composition;
(c) applying the buffered antibody composition to a blue dye column, e.g., a blue column described herein, under conditions that allow 80% or more of the hsIgG to flow through, but allows only 100 ppm or less of the ST6 or enzymatically active portion thereof to flow through, thereby producing purified hsIgG.

18. The method of claim 17, wherein the blue dye chromatography column is a Trisacryl Blue (TABS) column and the buffer suitable for use with the column is citrate buffer, about 100 mM, about pH 4.5, with about 800 mM NaCl.

19-20. (canceled)

21. The method of claim 17 , wherein the method does not contain any additional filtering, fractionation, or purification steps other than (a) and (b) before carrying out step (c).

22. The method of claim 17 , wherein the method comprises a single depth filtering step, in addition to steps (a) and (b) before step (c).

23-33. (canceled)

34. A method of preparing purified hypersialylated (hsIgG) composition, the method comprising
(a) providing a mixture of IgG antibodies,
(b) incubating the mixture of IgG antibodies in a reaction mixture comprising β1,4-Galactosyltransferase I (β4GalT) or enzymatically active portion thereof and UDP-Gal to produce galactosylated IgG antibodies;
(c) incubating the galactosylated IgG antibodies in a second reaction mixture comprising ST6Gal or enzymatically active portion thereof and CMP-NANA to produce a hsIgG mixture;
(d) applying the hsIgG mixture to a protein A column under conditions that bind IgG antibodies; and
(e) eluting hsIgG from the protein A column.

35. The method of claim 34, further comprising (f) further purifying the hsIgG produced in step (e) using a trisacryl blue column.

36. The method of claim 34, wherein step (e) comprises eluting the hsIgG with a buffer comprising glycine.

37. The method of claim 34, wherein the protein A column is washed with an acetate buffer between step (d) and step (e).

38. The method of claim 34, wherein the pH and salt content of hsIgG produced in step (e) is altered before applying it to the trisacryl blue column.

39. The method of claim 34, wherein step (f) comprises eluting the hsIgG with a high salt buffer.

40. The method of claim 39, wherein the high salt buffer comprises 2 M NaCl.

41. The method of claim 39, wherein the high salt buffer comprises 2 M KCl.

42. The method of claim 38, wherein the hsIgG produced in step (e) is altered to 0.4 M NaCl and pH 4.5.

43. (canceled)

44. The method of claim 34, wherein the B4GalT or enzymatically active portion thereof is at least 90% identical SEQ ID NO: 12 or SEQ ID NO. 13Error! Reference source not found. and the ST6Gal or enzymatically active portion thereof comprises an amino acid sequence that is at least 90% identical SEQ ID NO: 19 or SEQ ID NO: 20.

45-68. (canceled)

69. The method of claim 1 , wherein at least 60%, 65%, 70% of the branched glycans on the Fab domain of the hsIgG have a sialic acid on both the α 1,3 arm and the α 1,6 arm that is connected through a NeuAc-α 2,6-Gal terminal linkage after purification.

70-74. (canceled)

75. The method of claim 4, wherein at least 60%, 65%, 70%, 75%, 80%, or 85% of the branched glycans on the Fab domain of the hsIgG have a sialic acid on both the α 1,3 arm and the α 1,6 arm that is connected through a NeuAc-α 2,6-Gal terminal linkage after purification.

76. The method of claim 34, wherein at least 60%, 65%, 70%, 75%, 80%, or 85% of the branched glycans on the Fab domain of the hsIgG have a sialic acid on both the α 1,3 arm and the α 1,6 arm that is connected through a NeuAc-α 2,6-Gal terminal linkage after purification.
